(19) United States
(12) Patent Application Publication
(10) Pub. No.: US 2025/0223554 A1
(43) Pub. Date: Jul. 10, 2025

(54) METHODS AND COMPOSITIONS USING RECOMBINANT DENDRITIC CELLS FOR CANCER THERAPY

(71) Applicant: Renovaro Biopharma Inc., Los Angeles, CA (US)

(72) Inventors: Francois Binette, Los Angeles, CA (US); Elango Kathirvel, Los Angeles, CA (US); Lu Chen, Los Angeles, CA (US); Ariel Rodriquez, Los Angeles, CA (US); Jose D. Gonzalez, Los Angeles, CA (US); Neha Sheik, Los Angeles, CA (US); Mark Dybul, Los Angeles, CA (US)

(21) Appl. No.: 19/008,154

(22) Filed: Jan. 2, 2025

Related U.S. Application Data
(60) Provisional application No. 63/617,944, filed on Jan. 5, 2024.

(51) Int. Cl. C12N 5/0784; A61K 35/14; A61P 35/00; C12N 5/00; C12N 15/86

(52) U.S. Cl. CPC C12N 5/0639; A61K 35/14; A61P 35/00; C12N 5/562; C12N 15/86; C12N 2506/11; C12N 2510/00; C12N 2740/15043

(57) ABSTRACT

Disclosed herein are methods and compositions for treating cancer by eliciting an immune response by administering dendritic cells expressing heterologous proteins. In some embodiments, a dendritic cell comprises one or more heterologous nucleic acid molecules encoding for CD40L and CXCL13. In some embodiments, the dendritic cell further comprises a heterologous nucleic acid molecule encoding for CD93. In yet additional embodiments, the dendritic cells expressing heterologous proteins are activated.

### CROSS REFERENCE TO RELATED APPLICATIONS

The present application claims the benefit of U.S. Provisional Patent application 63/617,944 filed Jan. 5, 2024 which is incorporated by reference herein in its entirely.

### SEQUENCE LISTING

This application contains a sequence listing having filename 0440865.00143US_Sequence_Listing.xml, which is 16 KB in size, and was created on Dec. 26, 2024. The entire content of this sequence listing is incorporated herein by reference.

### BACKGROUND

Cancer therapies encompass a wide range of therapeutic approaches, including surgical, radiation, chemotherapy, as well as cell-based immunotherapy. While these various therapeutic approaches provide a broad selection of treatments, existing therapeutics suffer from many disadvantages, including a lack of selectivity of targeting cells over healthy cells, toxicity, and resistance by the cancer to the treatment. More recent approaches utilizing targeted therapeutics that interfere with key cellular processes of cancer cells preferentially over normal cells, have led to chemotherapeutic regimens with fewer side effects as compared to non-targeted therapies such as radiation treatment.

Cancer immunotherapy has also emerged as a promising therapeutic approach to augment and complement existing standards of care. These approaches include utilizing antibody to modulate the immune system to kill cancer cells. Anti-tumor immune responses in some patients with solid tumors have been enhanced with immunomodulating check point inhibitors such as anti-PD1 treatment. However, only a fraction of patients are responsive to such treatment, highlighting the need for additional approaches and further cancer treatments to augment and complement existing therapeutic standards of care.

### SUMMARY

Provided herein are dendritic cells comprising one or more heterologous nucleic acid molecules encoding for CD40L and/or CXCL13. In some embodiments, the one or more heterologous nucleic acid molecules further encodes for CD93.

In some embodiments, dendritic cells are genetically engineered to over-express one or more endogenous proteins including, but not limited to, CD40L, CXCL13, CD93, 4-1BBL, and/or IL-21.

Further provided herein is a dendritic cell comprising a heterologous CD40L protein and a heterologous CXCL13 protein. In some embodiments, the cell further comprises a heterologous CD93 protein.

In some embodiments, the dendritic cell is produced from a CD14+ monocyte. In some embodiments, the dendritic cell is produced from a CD34+ hematopoietic stem cell (HSC). In some embodiments, the HSC is, or is derived from, an embryonic stem cell or an induced pluripotent stem cell (iPSC).

Further provided herein are antigen-activated dendritic cells, wherein the dendritic cell comprises one or more heterologous nucleic acid molecules encoding for CD40L and/or CXCL13. In some embodiments, the one or more heterologous nucleic acid molecules further encodes for CD93. In some embodiments, the antigen activated dendritic cell is activated by exposure to one or more antigens. In some embodiments, the antigen is a tumor antigen. In some embodiments, the antigen is a viral antigen. In some embodiments, the antigen is a cell lysate. In some embodiments, the cell lysate is allogeneic or autologous to the antigen-activated dendritic cell. In some embodiments, the cell lysate is a lysate comprising one, or a combination of, allogeneic tumor cell lysates. In some embodiments, the cell lysate is a whole cell lysate. In some embodiments, the antigen is a purified tumor-associated antigen or a cancer neo-antigen. In some embodiments, the antigen is a synthetic antigen.

In some embodiments, the heterologous nucleic acid molecules are messenger ribonucleic acid (mRNA) molecules. In some embodiments, the heterologous nucleic acid molecules are comprised in an expression vector. In some embodiments, the mRNA is not associated with a vector. In some embodiments, the vector is a viral vector including, but not limited to a recombinant adenoviral vector, a recombinant retroviral vector, or a recombinant lentiviral vector, or a combination thereof. In some embodiments, the expression vector is a lentiviral vector. In some embodiments, heterologous nucleic acid molecule is associated with a transposon.

Further provided herein is a pharmaceutical composition comprising cells provided herein.

Further provided herein are methods of treating a solid tumor, cancer, or malignancy in a subject comprising administering to the subject any of cells or compositions provided for herein.

Further provided herein are methods of treating a solid tumor, cancer, or malignancy in a subject comprising administering to the subject any of cells or compositions provided for herein.

Also disclosed herein are methods of producing gene-modified dendritic cells for cancer therapy comprising: (a) obtaining CD34+ hematopoietic stem cells (HSCs); (b) expanding the CD34+ HSCs; (c) introducing one or more exogenous genes into the HSCs to produce gene-modified HSCs by infection with a viral vector comprising the genes; (d) differentiating the gene-modified HSCs into monocytes; (e) maturing the monocytes into immature dendritic cells (DCs); (f) maturing the immature Dcs into mature dendritic cells (MaDCs) to produce gene-modified (recombinant) MaDCs; and (g) harvesting the gene-modified MaDCs from the culture.

Further disclosed herein are methods of producing gene-modified dendritic cells for cancer therapy comprising: (a) obtaining CD34+ hematopoietic stem cells (HSCs); (b) expanding the CD34+ HSCs; (c) differentiating the gene-modified HSCs into monocytes; (d) maturing the monocytes into immature dendritic cells (DCs); (e) maturing the immature Dcs into mature dendritic cells (MaDCs) to produce gene-modified (recombinant) MaDCs; (f) introducing one or more exogenous genes into the HSCs to produce gene-modified HSCs by electroporation of mRNA encoding the genes; and (g) harvesting the gene-modified MaDCs from the culture.

In some embodiments, the method further comprises pulsing the gene-modified MaDCs with tumor antigens. In some embodiments, the tumor antigens are present in at least one tumor cell lysate. In some embodiments, the tumor cell lysate is prepared from a tumor or a tumor cell line. In some embodiments, the tumor cell lysate is allogeneic or autologous to the antigen activated dendritic cell. In some embodiments, the tumor antigens comprise a tumor tissue sample, a tumor cell line, a purified tumor antigen, or a synthetic tumor antigen.

In some embodiments, the immature dendritic cells are characterized by expression of CD209, HLA-DR, CD40, CD86 and CD14 and low expression of CCR7. In some embodiments, the mature dendritic cells are characterized by expression of CD1a, CCR7, HLA-DR and CD83 and low expression of CD14.

In some embodiments, the one or more exogenous genes are selected from CD93, CXCL13, CD40L, IL-21, and 4-1BBL. In some embodiments, the one or more exogenous genes comprise two genes, three genes, four genes, or five genes.

In some embodiments, the method further comprises cryopreserving the cells after one or more of steps (a)-(f).

Further disclosed hare methods of treating cancer, comprising administering to a subject in need thereof the gene-modified MaDCs prepared according to the methods disclosed herein.

In some embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma/colorectal cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the route of administration is via intratumoral, peritumoral, intradermal, subcutaneous, intramuscular, or intraperitoneal, injection. In some embodiments, the administering is via subcutaneous, intratumoral or intradermal injection. In some embodiments, the gene-modified MaDCs are cryopreserved and thawed prior to administration. In some embodiments, the MaDCs are not pulsed with an antigen prior to administration.

Also disclosed herein are kits comprising gene-modified MaDCs prepared by the methods disclosed herein, wherein the gene-modified MaDC are frozen or cryopreserved in a container, and optionally comprising a cryopreserved allogeneic tumor lysate in a separate container. In some embodiments, the gene-modified MaDC or composition is frozen in a container. In some embodiments, the kit further comprises separate containers comprising one or more buffers, and optionally comprising one or more activation agents. In some embodiments, the kit further comprises instructions for incubating and/or processing the dendritic cells or compositions.

Also disclosed herein are methods of treating cancer in a subject comprising administering to the subject gene-modified MaDCs prepared by the methods disclosed herein, wherein the gene-modified MaDCs or composition has not been pulsed with a tumor antigen.

Also disclosed herein are methods of treating cancer in a subject comprising administering to the subject gene-modified MaDCs prepared by the methods disclosed herein, wherein the gene-modified MaDCs has been pulsed with a tumor antigen or lysate.

Also disclosed herein are methods of activating the immune system, comprising administering to a subject in need thereof an effective amount of gene-modified MaDCs prepared by the methods disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts expression of heterologous molecules in monocyte-derived dendritic cells (DCs).

FIG. 2 depicts secretion of CXCL13 by gene-modified monocyte derived dendritic cells.

FIG. 3 depicts cell characteristics and expression of heterologous molecules after electroporation (EP) or no electroporation control (no electroporation).

FIG. 4A-H depict pancreatic tumors in mice before and after treatment with gene-modified DCs. FIG. 4A depicts a normal mouse pancreas. FIG. 4B depicts untreated established pancreatic tumors. FIG. 4C depicts established tumors treated with mature monocyte-derived DC electroporated with CD93/CXCL13/CD40L mRNA. FIG. 4D depicts established tumors treated with mature monocyte-derived MoDC electroporated with CD93/CXCL13/4-1BBL mRNA.

FIG. 4E depicts established tumors treated with mature monocyte-derived MoDC not electroporated with any heterologous molecules. FIG. 4F depicts established tumors treated with hematopoietic stem cell (HSC)-derived MaDC electroporated with CD93/CXCL13/CD40L mRNA.

FIG. 4G depicts established tumors treated with HSC-derived MaDC transduced with mock lentivirus. FIG. 4H depicts established tumors treated with HSC-derived MaDC transduced with a lentiviral vector expressing CD93/CXCL13/CD40L.

FIG. 5A-B depicts tumor weight (FIG. 5A) and tumor volume (FIG. 5B) of the tumors from FIG. 4.

FIG. 6A-D depicts depict pancreatic tumors in mice before and after treatment with gene-modified DCs exposed to tumor cell extracts. FIG. 6A depicts established untreated tumors. FIG. 6B depicts established tumors treated with MaDCs without gene modification. FIG. 6C depicts established tumors treated with gene-modified MaDCs exposed to MP2 cell extracts. FIG. 6D depicts established tumors treated with gene-modified MaDCs exposed to tumor extracts.

FIG. 7A-B depicts tumor weight (FIG. 7A) and tumor volume (FIG. 7B) of the tumors from FIG. 6.

### DETAILED DESCRIPTION

The present embodiments are directed, in part, to methods and compositions relating to dendritic cells heterologously expressing one or more of CD40L, CXCL13, CD93, 4-1BBL, and/or IL-21. In some embodiments, the CD40L, CXCL13, CD93, 4-1BBL, and/or IL-21 can be of human origin. In some embodiments, the CD40L, CXCL13, CD93, 4-1BBL, and/or IL-21 are of rodent (mouse or rat) or porcine origin. The cells comprising the heterologously-expressed proteins can be referred to as recombinant cells. The compositions can be a stand-alone recombinant DC (dendritic cell) composition, or in alternative embodiments the recombinant DC cells can be antigen activated by contacting the cells, which can also be referred to as “loading,” with tumor lysate (allogeneic or autologous) as a vaccination and/or immune adjuvant for subjects with solid tumors and malignancies. In some embodiments, the tumor lysate is allogeneic to the source of the dendritic cells. In some embodiments, the tumor lysate is autologous to the source of the dendritic cells. U.S. Pat. No. 11,413,338 entitled “Methods and compositions using recombinant dendritic cells for cancer therapy” is incorporated by reference herein for all it discloses regarding genetically engineered dendritic cells.

Dendritic cells (DCs) are professional antigen-processing cells. They have a number of receptors that enhance the uptake of antigens, and they are specialized to convert these antigens into major histocompatibility complex (MHC)-peptide complexes that can be recognized by lymphocytes.

In recent years it has been realized that an efficient manner of antigen delivery to T cells, especially to naive T cells, is by way of dendritic cells. Dendritic cells are the most efficient antigen presenting cells and DC-based immunotherapy have already been used in different settings for treatment of cancer (Kugler et al., 2000, Nat. Med. 6:332-336; Nestle et al., 1998, Nat. Med., 4:328-332; Thurner et al., 1999, J. Exp. Med., 190:1669-1678) demonstrating high potency of this way of immunization.

One of the unique properties of DCs is their ability to uptake exogenous proteins by endocytosis, which are then processed and presented as peptide epitopes on their surface in conjunction with MHC class I antigens. The antigen presenting dendritic cells can be recognized by cytotoxic T cells. This property is important when tumor cell antigens are applied in form of tumor lysates or apoptotic bodies added exogenously. High endocytic activity is believed to be associated with the immature state of DC differentiation based on comparison of immature and mature DC (Sallusto et al., 1995, J. Exp. Med., 182:389-400).

While not wishing to be bound by theory, the present compositions and methods are based at least in part on the ability of recombinant (or transgenic) dendritic cells to build a mixed leukocyte reaction (MLR) at the injection site. When tested in the MLR assay, the recombinant allogeneic DCs (alloDCs) described herein were the major stimulators and were unusually potent. In some embodiments, the DCs are allogeneic to the subject and are referred to as alloDCs.

In some embodiments, the DCs are transduced with CD40L and/or CXCL13 in order to maximize the attraction of patients' T cells and B cells, and generate the cascade of anti-tumor cellular and humoral immune responses. In some embodiments, the DCs are transduced with CD40L and CXCL13. In some embodiments, the DCs are transduced with CD40L, CXCL13, and CD93. In some embodiments, the DCs are transduced with CD40L and CD93. In some embodiments, the DCs are transduced with CXCL13 and CD93. In some embodiments, the cells are not transduced with CD93. In some embodiments, the DCs do not heterologously express CD93. In some embodiments, the DCs are also transduced with 4-1BBL and/or IL-21.

In some embodiments, monocytes, CD34+ HSCs, ESCs, or iPSCs, are infected with a viral vector encoding one or more heterologous nucleic acids disclosed herein. In some embodiments, monocytes are infected with a viral vector encoding one or more heterologous nucleic acids disclosed herein. In some embodiments, monocytes, CD34+ HSCs, ESCs, or iPSCs, are transduced with mRNA encoding one or more heterologous nucleic acids disclosed herein. In some embodiments, CD34+ HSCs are transduced with mRNA encoding one or more heterologous nucleic acids disclosed herein. In some embodiments, the monocytes, CD34+ HSCs, ESCs, or iPSCs are differentiated into dendritic cells prior to infection, or transduction, with one or more heterologous nucleic acids disclosed herein. In some embodiments, the dendritic cells transduced with mRNA encoding one or more heterologous nucleic acids disclosed herein are mature dendritic cells.

In optional further embodiments, transduction of the CD40L+ and CXCL13+ alloDCs with CD93 facilitates the cross-talk between the allogeneic DCs and the host DCs, creating a stable and sustained host anti-tumor immunogenicity.

In some embodiments, monocytes or hematopoietic stem cells (HSCs) are transfected with a recombinant viral vector encoding for CD40L and CXCL13 (and optionally CD93, 4-1BBL, and/or IL-21), or any, combination thereof as described herein, to be stably expressed on the cells. In some embodiments, mRNA encoding for CD40L and CXCL13 (and optionally CD93, 4-1BBL, and/or IL-21) are introduced into monocytes, HSCs, or dendritic cells via electroporation Positively transduced cells can be selected and the cells can then be differentiated into immature and/or mature DCs in vitro. In some embodiments, the recombinant DCs are exposed to tumor antigens before administration to a subject. For the resected and biopsied malignant patients, the autologous tumor lysate can be generated and the tumor lysate can be presented to the immature recombinant alloDCs (recombinant im alloDCs) for the autologous tumor lysate to be processed by the recombinant im alloDCs. In some embodiments, the protein expression profile of the resected tumor samples and biopsy samples are screened. For any non-resectable and non-biopsiable patient, the recombinant CD40L+CXCL13+ alloDC (or optionally further including CD93+, 4-1BBL+, and/or IL-21+) can be administered to the patient without antigen loading as an immune adjuvant. Using this CD40L+CXCL13+ (optionally CD93, 4-1BBL, and/or IL-21) alloDC approach, late stage cancer patients can mount new antitumor cellular and humoral immune response to tumor antigens and, without being bound to any particular mechanism, enhance the immune response.

In some embodiments, the source of dendritic cells can be from donor peripheral blood, and includes dendritic cells derived from CD14+ monocytes or CD34+ HSCs. In some embodiments, the donor is not the same as the subject being treated with the dendritic cells and compositions described herein. In some embodiments, the source of dendritic cells is from leukophoresis. In some embodiments, the source of dendritic cells is embryonic stem cells. In some embodiments, the source of dendritic cells is induced pluripotent stem cells.

In some embodiments, the recombinant dendritic cells are treated with interferon-alpha (IFN-α) and/or 5′-aza-2′deoxycytidine (Aza) before or during loading the recombinant dendritic cells with tumor cell lysate/and or antigen. See, https://doi.org/10.1101/531616.

In some embodiments, isolated monocytes or HSCs are transduced with a recombinant vector, such as an adenoviral or lentiviral vector, comprising human CD40L and CXCL13, and optionally also CD93, 4-1BBL, and/or IL-21 constructs, allowing the recombinant molecules to be expressed on isolated human monocytes or HSCs. In some embodiments, dendritic cells are electroporated with mRNA encoding human CD40L and CXCL13, and optionally also CD93, 4-1BBL, and/or IL-21. The recombinant cells are then selected or enriched by a positive isolation for CD93 or in some cases without selection or enrichment. These recombinant cells are then differentiated into immature DCs (imDC) followed by mature DCs (maDC) in vitro, and are referred to a recombinant allogeneic DCs (recombinant alloDCs). In some embodiments, the expression is stable expression. In some embodiments, the expression is over-expression. “Over-expression” refers to a level that is at least 10%, 20%, 30%, 40%, 50%, 60%, 70%, 80%, 90%, 100%, 200%, 300%, 400%, or 500% over native expression levels. Native expression levels refer to expression levels of the cells that have not been transduced with a vector to express the heterologous protein or proteins of interest, such as CD40L, CXCL13, and/or CD93.

In some embodiments, the DCs (recombinant or unmodified) are matured by the contacting the cells with maturation factors after loading with tumor cell lysate and/or antigen. Such maturation factors include at least: IL-1B, IL-6, TNF-α, and prostaglandin E2 (PGE2), or any combination thereof. In some embodiments, the DCs are matured by the contacting the cells with maturation factors prior to being contacted with tumor cell lysate and/or antigen. In some embodiments, the DCs are matured with the maturation factors and are not contacted with tumor cell lysate and/or antigen. In some embodiments, dendritic cells are cultured or incubated with a cytokine selected from the group consisting of IL-4, GM-CSF, IL-13, IFN-γ, Flt-31, stem cell factor (SCF), and TNF-α.

In some embodiments, monocytes, HSCs, ESCs, or iPSCs are genetically engineered to over-express endogenously expressed genes including, but not limited to, CD40L and CXCL13, and optionally also CD93, 4-1BBL, and/or IL-21. In some embodiments, endogenously genes are overexpressed by methods including, but not limited to, delivering CRISPR/Cas system, TALENS, and zinc-finger nucleases.

### Definitions

As used in this application and in the claims, the singular forms “a,” “an,” and “the” include the plural forms unless the context clearly dictates otherwise. Additionally, the term “includes” means “comprises.”

As used herein, the term “transduced” refers cells which have been exposed to heterologous proteins by electroporation, viral vector infection, or through a method such as the CRISPR/Cas system. The term “transduced” is not limiting to a specific method of introducing genes.

The term “patient” and “subject” are interchangeable and may be taken to mean any living organism which may be treated with compounds of the present disclosure. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein. Additionally, if a range is written as “about X to Y” the “about” modifies both the X and the Y values unless context indicates otherwise.

The terms “administer,” “administering” or “administration” as used herein refer to either directly administering a compound, cell, composition, or pharmaceutical composition, which can also referred to as an agent of interest. In some embodiments, the compositions have been sterilized or filtered to remove any viral or bacterial particles.

The terms “co-administration” or the like, are meant to encompass administration of the selected therapeutic agents to a single patient, and are intended to include treatment regimens in which the agents are administered by the same or different route of administration or at the same or different time.

A “therapeutically effective amount” of a composition is an amount sufficient to achieve the desired effect, i.e., to ameliorate, prevent or improve an unwanted condition, disease or symptom of a patient. The activity contemplated by the present methods may include both therapeutic and/or prophylactic treatment, as appropriate. The specific dose can be determined by the particular circumstances surrounding the case, including, for example, the therapeutic administered, the route of administration, and the condition being treated. The effective amount administered may be determined by a physician in the light of the relevant circumstances, including the condition to be treated, the choice of the therapeutic to be administered, and the chosen route of administration.

The term “inhibit” includes the administration of a therapeutic of embodiments herein to prevent the onset of the symptoms, alleviating the symptoms, or eliminating the disease, condition or disorder.

By “pharmaceutically acceptable”, it is meant the carrier, diluent or excipient must be compatible with the other ingredients of the therapeutic and not deleterious to the recipient thereof.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. Beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In embodiments or claims where the term comprising is used as the transition phrase, such embodiments can also be envisioned with replacement of the term “comprising” with the terms “consisting of” or “consisting essentially of.”

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies may exist in a variety of forms including, for example, polyclonal antibodies, monoclonal antibodies, Fv, Fab, and F(ab)₂, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

The term “antigen” as used herein is defined as a molecule that provokes an immune response. This immune response may involve either antibody production, or the activation of specific immunologically-competent cells, or both. The antigen can also be used in vitro to activate the DCs, which is comparable to an immune response. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the embodiments include, but are not limited to, the use of partial nucleotide sequences of more than one gene and that these nucleotide sequences are arranged in various combinations to elicit the desired immune response. In some embodiments, the antigen is a purified tumor-associated antigen or a cancer neo-antigen. In some embodiments, the antigen is a synthetic antigen. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. As described herein, the antigen can be a tumor cell lysate. Examples of tumor cell lysates include, but are not limited to, those described herein, lysates prepared from tumor biopsies or tumor resections. Methods of preparing lysates are known and any method can be used.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

Cancers that may be treated include tumors that are not vascularized, or not yet substantially vascularized, as well as vascularized tumors. Types of cancers to be treated with the recombinant dendritic cells described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The term “anti-tumor effect” as used herein, refers to a biological effect that can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the recombinant cells and therapeutic compositions to prevent the occurrence of tumor in the first place.

As used herein, the term “autologous” is meant to refer to any material derived from the same individual to which it is later to be re-introduced into the individual.

“Allogeneic” refers to a sample (graft, cell, or population of cells) derived from a different animal of the same species to which the sample is later introduced. In some embodiments, the recombinant dendritic cells are completely HLA mismatched as compared to the subject receiving the recombinant dendritic cells, unlike the situations in renal and bone marrow transplantations where matching HLA-A, -B, and -DR are beneficial for graft survival. In the present methods for treating cancer and/or increasing and/or improving the immune response, the recombinant dendritic cells expressing CD40L, CXCL13, and optionally CD93, 4-1BBL, and/or IL-21 can attract T cells and, for example, generate humoral immune responses, which serve to target the cancer cells and bind to them, and kill them, releasing tumor cell antigens. This cycle repeats itself with the allogeneic recombinant dendritic cells, until they are killed by the host cell immune responses. This can happen, for example, within about 4 to about 7 days (and possibly during an extended range of about 4 to about 15 days). Thus, there is reduced or no risk for GVHD (graft-versus-host disease) with such a short life-span for the allogeneic recombinant DCs. However, in the process of recognizing and killing tumor cells and releasing antigens for that span of about 4 to about 7 days, the allogeneic cells can, in some embodiments, serve to vaccinate the host dendritic cells and other immune cells to recognize and kill the tumor cells. See additionally, review article Cancer Sci. 2019 110:16-22, which is hereby incorporated by reference in its entirety. The induction of anti-tumor immunity is a cyclic process that can be self-propagating. It can amplify and extend T cell responses against cancer cells. It also contains several inhibitory factors itself to halt the cycle when the target cells (cancer cells) are eradicated. The cycle can be divided into seven steps, starting with the release of tumor antigens from the cancer cells and ending with the killing of cancer cells. APC refers to antigen-presenting cell and DC refers to dendritic cell.

A “disease” is a state of health of a subject wherein the subject cannot maintain homeostasis, and wherein if the disease is not ameliorated then the animal's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. As used herein, a vector that encodes a protein of interest refers to a vector containing a nucleotide sequence that encodes for that protein or proteins.

As used herein, the term “exogenous” refers to any material introduced from or produced outside an organism, cell, tissue or system. A protein that is referred to as a heterologous protein is expressed by exogenous material (vectors, nucleotide sequences, and the like) that has been introduced into the organism, cell, tissue or system. For the avoidance of doubt, a heterologous protein, a heterologous vector, or heterologous nucleotide molecule is not the same as that may present in the native, unmodified genome of the organism, cell, tissue or system. A heterologous protein, vector, or nucleotide sequence that may have the same or similar to a sequence already present in the organism, cell, tissue, or system, is expressed from a location or sequence that is other than the native sequence found in the genome of that organism, cell, tissue, or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” or “Homology” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. Generally, a comparison is made when two sequences are aligned to give maximum homology.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated” biological component (such as a nucleic acid, protein or cell) has been substantially separated or purified away from other biological components (such as cell debris, other proteins, nucleic acids or cell types). Biological components that have been “isolated” include those components purified by standard purification methods.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Chemotherapy includes treatment with a chemical agent (such as a cytotoxic agent) with therapeutic utility for treating diseases characterized by abnormal cell growth, such as tumors, neoplasms, cancer and psoriasis. Examples of chemotherapies include, but are not limited to those described herein.

As used herein, recombinant generally refers to the following: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

As used herein, the following abbreviations for the commonly occurring nucleic acid bases are used. “A” refers to adenosine, “C” refers to cytosine, “G” refers to guanosine, “T” refers to thymidine, and “U” refers to uridine.

The term “leukocytes” or “white blood cell” as used herein refers to any immune cell, including monocytes, neutrophils, eosinophils, basophils, and lymphocytes. The term “lymphocytes” as used herein refer to cells commonly found in lymph, and include natural killer cells (NK cells), T-cells, and B-cells. It will be appreciated by one of skill in the art that the above listed immune cell types can be divided into further subsets.

The term “tumor infiltrating leukocytes” as used herein refers to leukocytes that are present in a solid tumor.

The term “blood sample” as used herein refers to any sample prepared from blood, such as plasma, blood cells isolated from blood, and so forth.

The term “purified sample” as used herein refers to any sample in which one or more cell subsets are enriched. A sample may be purified by the removal or isolation of cells based on characteristics such as size, protein expression, and so forth.

As used herein, immunodeficient means lacking in at least one essential function of the immune system. As used herein, an “immunodeficient” subject (such as a human) is one lacking specific components of the immune system or lacking function of specific components of the immune system (such as, for example, B cells, T cells, NK cells or macrophages). In some cases, an immunodeficient subject comprises one or more genetic alterations that prevent or inhibit the development of functional immune cells (such as B cells, T cells or NK cells). In some examples, the genetic alteration is in IL17 or IL17 receptor.

As used herein, immunosuppressed refers to a reduced activity or function of the immune system. A subject can be immunosuppressed, for example, due to treatment with an immunosuppressant compound or as a result of a disease or disorder (for example, immunosuppression that results from HIV infection or due to a genetic defect). In some cases, immunosuppression occurs as the result of a genetic mutation that prevents or inhibits the development of functional immune cells, such as T cells.

Examples of types of cancer and proliferative disorders that can be treated with an effective amount of recombinant dendritic cellular compositions and related methods described herein include, but are not limited to, fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilm's tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. The treatment and/or prevention of cancer includes, but is not limited to, alleviating one or more symptoms associated with cancer, the inhibition or reduction of the progression of cancer, the promotion of the regression of cancer, and/or the promotion of the immune response.

In certain other embodiments, a “therapeutically effective amount” is the amount of the recombinant dendritic cellular composition that results in a reduction of the tumor, growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition or cells described herein relative to the tumor growth or spread of cancer in a patient (or an animal) or a group of patients (or animals) not administered a composition or cells of the present disclosure.

### Dendritic Cell Compositions

Disclosed herein are methods and compositions for treating cancer by eliciting an immune response by administering dendritic cells expressing heterologous proteins. In some embodiments, the DCs heterologously express CD40L and CXCL13. In some embodiments, the DCs heterologously express CD40L, CXCL13, and CD93. In some embodiments, the DCs heterologously express CD40L and CD93. In some embodiments, the DCs heterologously express CXCL13 and CD93. In some embodiments, the DCs do not heterologously express CD93. In some embodiments, the DCs further heterologously express one or more of 4-1BBL and/or IL-21.

In some embodiments the dendritic cells are allogeneic as to the subject they are administered to, and as such, are not restricted by HLA expression. The allogeneic nature of the cells as compared to the subject is an added advantage to mount an improved anti-tumor effect.

In some embodiments, the dendritic cells are autologous to the subject that they are administered to.

Thus, while not wishing to be bound by theory, aspects of the present disclosure relates to providing dendritic cells expressing heterologous proteins (e.g. recombinant alloDCs) to a patient, to elicit an improved immune response to cancer (tumor) in the patient. The methods and compositions described herein have many advantages over previous compositions and methods, including the lack of co-culturing on feeder cells, as well as to minimize risks of contamination of the cellular composition due to the short in vitro culture/incubation time for recombinant alloDC and optionally loading with autologous or “off the shelf” antigens, and ease and speed of preparing and delivering the cellular composition to the patient, along with very minimal side effects from the cellular composition, which are typically only Grade 1 (or less side effects). These recombinant allo-DC cells and compositions provide the added benefit of eliciting both cellular and humoral immune responses in the patient. Furthermore, the host alloreactivity against the HLA-mismatched DCs amplifies the tumor-specific immune response. These methods also serve to provide a “vaccine effect” training the host, patient immune cells to similarly recognize and kill the cancer target, and boosting the host cellular and humoral responses. In some embodiments, the subject receiving the cells views the cells as foreign and will mount an immune response against the cells, which could be referred to as rejecting the cells. This “rejection” can amplify the tumor-specific immune response in the subject.

In some embodiments, the recombinant dendritic cells are administered to a subject in need thereof with a pharmaceutically acceptable carrier in the form of a compositions.

The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, and additional pharmaceutical agents, and is incorporated by reference herein in its entirety.

In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the recombinant dendritic cellular compositions can be administered simultaneously with anti-microbial, anti-viral and other therapeutic agents. Alternatively, recombinant dendritic cellular compositions can be administered at selected times in advance of times when anti-microbial, anti-viral and other therapeutic agents are administered.

### Combinations

In some aspects, any of the methods of treatment described herein can further comprise administering one or more additional anti-cancer therapies to the individual. Various classes of anti-cancer agents can be used. Non-limiting examples include: radiation therapy, alkylating agents (e.g. cisplatin, carboplatin, or oxaliplatin), antimetabolites (e.g., azathioprine or mercaptopurine), anthracyclines, plant alkaloids (including, e.g. vinca alkaloids (such as, vincristine, vinblastine, vinorelbine, or vindesine) and taxanes (such as, paclitaxel, taxol, or docetaxel)), topoisomerase inhibitors (e.g., camptothecins, irinotecan, topotecan, amsacrine, etoposide, etoposide phosphate, or teniposide), podophyllotoxin (and derivatives thereof, such as etoposide and teniposide), antibodies (e.g., monoclonal or polyclonal), tyrosine kinase inhibitors (e.g., imatinib mesylate (GLEEVEC® or GLIVEC®)), hormone treatments, soluble receptors and other antineoplastics (e.g., dactinomycin, doxorubicin, epirubicin, bleomycin, mechlorethamine, cyclophosphamide, chlorambucil, or ifosfamide).

Additionally, in some embodiments, the cells and compositions provided herein can be used adjunctive to, or with, other agents or treatments having anti-cancer properties (See, U.S. Pat. No. 9,914,783, which is hereby incorporated by reference in its entirety). When used adjunctively, the recombinant DCs and related compositions and other agent(s) may be formulated together in a single, combination pharmaceutical formulation, or may be formulated and administered separately, either on a single coordinated dosing regimen or on different dosing regimens. Agents administered adjunctive to or with the recombinant DCs and related compositions will typically have complementary activities to the recombinant DCs and related compositions, such that the cells and other agents do not adversely affect each other.

Agents that may be used adjunctively with anti-PD-1 antibodies include, but are not limited to, alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, and the like, as well as combinations of one or more of these agents.

BiTE antibodies are bispecific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B.

siRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′—OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. Multivalent binding proteins are engineered to have the two or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

In some embodiments, the recombinant dendritic cellular compositions can be administered simultaneously with antibodies specific for a selected cancer type. Alternatively, recombinant dendritic cellular compositions can be administered at selected times in advance of times when antibodies specific for a selected cancer type are administered. Antibodies specific for a selected cancer type include any antibody approved for treatment of cancer. Examples include trastuzumab (HERCEPTIN®) for breast cancer, rituximab (RITUXAN®) for lymphoma, and cetuximab (ERBITUX®) for head and neck squamous cell carcinoma.

Additional examples of such antibody agents include inhibitors of PD-1 or PD-L1 (B7-H1), such as anti-PD-1 antibodies, including nivolumab (NIVOLUMAB®, Bristol-Myers Squibb) and pembrolizumab/lambrolizumab, also known as MK-3475 (KEYTRUDA®, Merck), pidilizumab (Curetech), AMP-224 (Amplimmune), and anti-PD-L1 antibodies, including MPDL3280A (Roche), MDX-1105 (Bristol-Myers Squibb), MEDI-4736 (AstraZeneca), and MSB-0010718 C (Merck). Other checkpoint inhibitors include antagonists of CTLA-4, such as anti-CTLA-4 antibodies. An exemplary anti-CTLA4 antibody is YERVOY® (ipilimumab, Bristol-Myers Squibb). Other exemplary CTLA-4 antibodies include tremelimumab (Pfizer), ticilimumab (AstraZeneca), and AMGP-224 (Glaxo SmithKline). Combinations with any two of these antibodies may also be indicated in certain instances.

Alkylating agents include, but are not limited to, altretamine, AMD-473, AP-5280, apaziquone, bendamustine, brostallicin, busulfan, carboquone, carmustine e (BCNU), chlorambucil, CLORETAZINE® (laromustine, VNP 40101M), cyclophosphamide, dacarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, and trofosfamide.

Angiogenesis inhibitors include, but are not limited to, endothelial-specific receptor tyrosine kinase (Tie-2) inhibitors, epidermal growth factor receptor (EGFR) inhibitors, vascular endothelial growth factor receptor (VEGF) inhibitors, delta-like ligand 4 (DLL4) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, and vascular endothelial growth factor receptor tyrosine kinase (VEGFR) inhibitors.

Antibody drug conjugates include, but are not limited to, those that target c-Met kinase (e.g., ADCs described in U.S. Pat. No. 7,615,529), LRRC15, CD30 (e.g., ADCETRIS® (brentuximab vedotin)), CS1 (e.g., ADCs described in US publication no. 20160122430), DLL3 (e.g., rovalpituzumab tesirine (ROVA-T)), HER2 (e.g., KADCYLA® (trastuzumab emtansine)), EGFR (e.g., ADCs described in US publication no. 20150337042), and prolactin receptor (e.g., ADCs described in US publication no. 20140227294).

Antimetabolites include, but are not limited to, ALIMTA® (pemetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, ribavirin, triapine, trimetrexate, S-1, tiazofurin, tegafur, TS-1, vidarabine, and UFT.

Antivirals include, but are not limited to, ritonavir, acyclovir, cidofovir, ganciclovir, foscarnet, zidovudine, ribavirin, and hydroxychloroquine.

Aurora kinase inhibitors include, but are not limited to, ABT-348, AZD-1152, MLN-8054, VX-680, Aurora A-specific kinase inhibitors, Aurora B-specific kinase inhibitors and pan-Aurora kinase inhibitors.

Bcl-2 protein inhibitors include, but are not limited to, ABT-263 (navitoclax), AT-101 ((−) gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl) piperazin-1-yl)benzoyl)-4-(((1R)-3-(dimethylamino)-1-((phenyl sulfanyl)methyl) propyl)amino)-3-nitrobenzene sulfonamide), N-(4-(4-((2-(4-chlorophenyl)-5,5-dimethyl-1-cyclohex-1-en-1-yl)methyl) piperazin-1-yl)benzoyl)-4-(((1R)-3-(morpholin-4-yl)-1-((phenylsulfanyl)methyl-) propyl)amino)-3-((trifluoromethyl) sulfonyl)benzenesulfonamide, venetoclax and GX-070 (obatoclax).

Bcr-Abl kinase inhibitors include, but are not limited to, DASATINIB® (BMS-354825) and GLEEVEC® (imatinib).

BTK inhibitors include, but are not limited to, ibrutinib and acalabrutinib.

CDK inhibitors include, but are not limited to, AZD-5438, BMI-1040, BMS-032, BMS-387, CVT-2584, flavopyridol, GPC-286199, MCS-5A, PD0332991, PHA-690509, seliciclib (CYC-202, R-roscovitine), abemaciclib, palbociclib, and ZK-304709.

COX-2 inhibitors include, but are not limited to, ABT-963, ARCOXIA® (etoricoxib), BEXTRA® (valdecoxib), BMS347070, CELEBREX® (celecoxib), COX-189 (lumiracoxib), CT-3, DERAIVIAXX® (deracoxib), JTE-522, 4-methyl-2-(3,4-dimethylphenyl)-1-(4-sulfamoylphenyl-1H-pyrrole)-, MK-663 (etoricoxib), NS-398, parecoxib, RS-57067, SC-58125, SD-8381, SVT-2016, S-2474, T-614, and VIOXX® (rofecoxib).

EGFR inhibitors include, but are not limited to, ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TAGRISSO® (osimertinib), TP-38, EGFR fusion protein, and TYKERB® (lapatinib).

ErbB2 receptor inhibitors include, but are not limited to, CP-724-714, CI-1033 (canertinib), Herceptin® (trastuzumab), TYKERBR (lapatinib), OMNITARG® (2C4, pertuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecific antibodies, mAB AR-209, and mAB 2B-1.

Histone deacetylase inhibitors include, but are not limited to, depsipeptide, LAQ-824, MS-275, trapoxin, suberoylanilide hydroxamic acid (SAHA), TSA, and valproic acid.

HSP-90 inhibitors include, but are not limited to, 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FC1, PU-3, radicicol, SNX-2112, STA-9090, and VER49009.

Inhibitors of apoptosis proteins include, but are not limited to, HGS1029, GDC-0145, GDC-0152, LCL-161, and LBW-242.

Activators of death receptor pathway include, but are not limited to, TRAIL, antibodies or other agents that target TRAIL or death receptors (e.g., DR4 and DR5) such as Apomab, conatumumab, ETR2-ST01, GDC0145 (lexatumumab), HGS-1029, LBY-135, PRO-1762, and trastuzumab.

Kinesin inhibitors include, but are not limited to, Eg5 inhibitors such as AZD4877, ARRY-520, and CENPE inhibitors such as GSK923295A.

JAK-2 inhibitors include, but are not limited to, CEP-701 (lesaurtinib), XL019, and INCB018424.

MEK inhibitors include, but are not limited to, ARRY-142886, ARRY-438162, PD-325901, and PD-98059.

mTOR inhibitors include, but are not limited to, AP-23573, CCI-779, everolimus, RAD-001, rapamycin, temsirolimus, ATP-competitive TORC1/TORC2 inhibitors, including PI-103, PP242, PP30, and Torin 1.

Non-steroidal anti-inflammatory drugs include, but are not limited to, AMIGESIC® (salsalate), DOLOBID® (diflunisal), Motrin® (ibuprofen), Orudis® (ketoprofen), Relafen® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), and DAYPRO® (oxaprozin).

PDGFR inhibitors include, but are not limited to, C-451, CP-673, and CP-868596.

Platinum chemotherapeutics include, but are not limited to, cisplatin, ELOXATIN® (oxaliplatin) eptaplatin, lobaplatin, nedaplatin, PARAPLATIN® (carboplatin), satraplatin, and picoplatin.

Polo-like kinase inhibitors include, but are not limited to, BI-2536.

Phosphoinositide-3 kinase (PI3K) inhibitors include, but are not limited to, wortmannin, LY294002, XL-147, CAL-120, ONC-21, AEZS-127, ETP-45658, PX-866, GDC-0941, BGT226, BEZ235, and XL765.

Thrombospondin analogs include, but are not limited to, ABT-510, ABT-567, ABT-898, and TSP-1.

VEGFR inhibitors include, but are not limited to, ABT-869, AEE-788, ANGIOZYME™ (a ribozyme that inhibits angiogenesis), axitinib (AG-13736), AZD-2171, CP-547,632, CYRAMZA® (ramucirumab), IM-862, MACUGEN® (pegaptamib), NEXAVAR® (sorafenib, BAY43-9006), pazopanib (GW-786034), vatalanib (PTK-787, ZK-222584), Sutent® (sunitinib, SU-11248), STIVARGA® (regorafenib), VEGF trap, and ZACTIMA™ (vandetanib, ZD-6474).

Antibiotics include, but are not limited to, intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), and zinostatin.

Topoisomerase inhibitors include, but are not limited to, aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCER or PHARMORUBICIN® (epirubicin), etoposide, exatecan, 10-hydroxycamptothecin, gimatecan, lurtotecan, mitoxantrone, ONIVYDE™ (liposomal irinotecan), orathecin, pirarbucin, pixantrone, rubitecan, sobuzoxane, SN-38, tafluposide, and topotecan.

Antibodies include, but are not limited to, AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, OX-40 specific antibodies, PANOREX® (edrecolomab), RENCAREX® (WX G250), RITUXAN® (rituximab), ticilimumab, trastuzumab, pertuzumab, VECTIBIX® (panitumumab), and CD20 antibodies types I and II.

Hormonal therapies include, but are not limited to, ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL® (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, Megace® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (histrelin implant), VETORYL® (trilostane or modrastane), and ZOLADEX® (fosrelin, goserelin).

Deltoids and retinoids include, but are not limited to, seocalcitol (EB1089, CB1093), lexacalcitrol (KH1060), fenretinide, PANRETIN® (aliretinoin), ATRAGEN® (liposomal tretinoin), Targretin® (bexarotene), and LGD-1550.

PARP inhibitors include, but are not limited to, ABT-888 (veliparib), KU-59436, AZD-2281 (olaparib), AG-014699 (rucaparib), MK4827 (niraparib), BMN-673 (talazoparib), iniparib, BSI-201, BGP-15, INO-1001, and ONO-2231.

Plant alkaloids include, but are not limited to, vincristine, vinblastine, vindesine, and vinorelbine.

Proteasome inhibitors include, but are not limited to, VELCADE® (bortezomib), KYPROLIS® (carfilzomib), MG132, NPI-0052, and PR-171.

Examples of immunologicals include, but are not limited to, interferons, immune checkpoint inhibitors, co-stimulatory agents, and other immune-enhancing agents. Interferons include interferon alpha, interferon alpha-2a, interferon alpha-2b, interferon beta, interferon gamma-1a, ACTIMMUNE® (interferon gamma-1b) or interferon gamma-n1, combinations thereof and the like. Immune check point inhibitors include antibodies that target PD-L1 (e.g., durvalumab, atezolizumab, avelumab, MEDI4736, MSB0010718C and MPDL3280A), and CTLA4 (cytotoxic lymphocyte antigen 4; e.g., ipilimumab, tremelimumab). Co-stimulatory agents include, but are not limited to, antibodies against CD3, CD40, CD40L, CD27, CD28, CSF1R, CD137 (e.g., urelumab), B7H1, GITR, ICOS, CD80, CD86, OX40, OX40L, CD70, HLA-DR, LIGHT, LIGHT-R, TIM3, A2AR, NKG2A, KIR (e.g., lirilumab), TGF-β (e.g., fresolimumab), and combinations thereof.

Other agents include, but are not limited to, ALFAFERONE® (IFN-α), BAM-002 (oxidized glutathione), BEROMUN® (tasonermin), BEXXAR® (tositumomab), CAMPATH® (alemtuzumab), dacarbazine, denileukin, epratuzumab, GRANOCYTE® (lenograstim), lentinan, leukocyte alpha interferon, imiquimod, melanoma vaccine, mitumomab, molgramostim, MYLOTARG™ (gemtuzumab ozogamicin), NEUPOGEN® (filgrastim), OncoVAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (tetrachlorodecaoxide (TCDO)), Proleukin® (aldesleukin), ZADAXIN® (thymalfasin), ZINBRYTA® (daclizumab high-yield process), and ZEVALIN® (90Y-Ibritumomab tiuxetan).

Biological response modifiers are agents that modify defense mechanisms of living organisms or biological responses, such as survival, growth or differentiation of tissue cells to direct them to have anti-tumor activity and include, but are not limited to, krestin, lentinan, sizofiran, picibanil PF-3512676 (CpG-8954), and ubenimex.

Pyrimidine analogs include, but are not limited to, cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), and TROXATYL™ (triacetyluridine troxacitabine).

Purine analogs include, but are not limited to, LANVIS® (thioguanine) and PURINETHOL® (mercaptopurine).

Antimitotic agents include, but are not limited to, batabulin, epothilone D (KOS-862), N-(2-((4-hydroxyphenyl)amino)pyridin-3-yl)-4-methoxybenzenesulfonamide, ixabepilone (BMS 247550), TAXOL® (paclitaxel), TAXOTERE® (docetaxel), PNU100940 (109881), patupilone, XRP-9881 (larotaxel), vinflunine, and ZK-EPO (synthetic epothilone).

Ubiquitin ligase inhibitors include, but are not limited to, MDM2 inhibitors, such as nutlins, and NEDD8 inhibitors such as MLN4924.

The recombinant DCs and related compositions may also be used to enhance the efficacy of radiation therapy. Examples of radiation therapy include external beam radiation therapy, internal radiation therapy (i.e., brachytherapy), and systemic radiation therapy.

The recombinant DCs and related compositions may be administered adjunctive to or with other chemotherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); O: Vincristine (ONCOVIN®); P: prednisone), CYPAT™ (cyproterone acetate), combrestatin A4P, DAB (389) EGF (catalytic and translocation domains of diphtheria toxin fused via a His-Ala linker to human epidermal growth factor) or TRANSMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EP0906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, hi strelin, hydroxycarbamide, ibandronic acid, IGN-101, IL-13-PE38, IL-13-PE38QQR (cintredekin besudotox), IL-13-pseudomonas exotoxin, interferon-alpha, interferon-gamma, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASER (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paclitaxel, PANDIMEX™ (aglycone saponins from ginseng comprising 20 (S) protopanaxadiol (aPPD) and 20 (S) protopanaxatriol (aPPT)), panitumumab, PANVAC®-VF (investigational cancer vaccine), pegaspargase, PEG Interferon A, phenoxodiol, procarbazine, rebimastat, REMOVABR (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPER (STn-KLH), thymitaq (2-amino-3,4-dihydro-6-methyl-4-oxo-5-(4-pyridylthio) quinazoline dihydrochloride), TNFERADE™ (adenovector: DNA carrier containing the gene for tumor necrosis factor-alpha), TRACLEER® or ZAVESCAR (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), and zorubicin, as well as combinations of any of these agents.

### Cell Sources

In some embodiments, dendritic cells are isolated from, or derived from, various sources, including peripheral blood mononuclear cells, bone marrow, HSCs, ESCs, and iPSCs.

“Peripheral blood mononuclear cells,” “PBMCs” or “mononuclear cells” refer to mononuclear cells separated from peripheral blood typically used for anti-cancer immunotherapy. The PBMCs can be obtained from human blood collected using known methods such as the Ficoll-Hypaque density gradient method.

According to one exemplary embodiment, “PBMCs” may be obtained from any suitable person. The source of the donor cells, including sources such as PBMCs, as used herein, can in some embodiments, be allogeneic to the recipient patient for isolation of the dendritic cells for use in the cancer treatment and immune stimulating methods described herein. Thus, in some embodiments, the donor inhibitory ligand mismatches with the patient (e.g. host) HLA.

In alternative embodiments, the source of the donor cells as used herein are autologous to the recipient patient for isolation of the dendritic cells for use in the cancer treatment and immune stimulating methods described herein.

PBMCs can be isolated by Ficoll-Hypaque density gradient centrifugation of samples obtained from discarded, de-identified leukocyte reduction filters, or blood donations from healthy volunteers with informed consent. Descriptions of cell populations, sources and methods for selecting or enriching for desired cell types can be found, for example in: U.S. Pat. No. 9,347,044, which is incorporated by reference in its entirety. Populations of cells for use in the methods described herein for treating mammals must be species matched, such as human cells. The cells may be obtained from an animal, e.g., a human patient. If the cells are obtained from an animal, they may have been established in culture first, e.g., by transformation; or more preferably, they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these. In an illustrative embodiment, a cell population is subjected to negative selection for depletion of non-T cells and/or particular T cell subsets. Negative selection can be performed on the basis of cell surface expression of a variety of molecules, including B cell markers such as CD19, and CD20; monocyte marker CD14; the NK cell marker CD56. Alternately, a cell population may be subjected to negative selection for depletion of non-CD34+ hematopoietic cells and/or particular hematopoietic cell subsets. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

Populations of cells include peripheral blood mononuclear cells, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

Methods of obtaining populations of cells comprising a monocytes (to be matured into DCs) or dendritic cells are well known in the art. For example, peripheral blood mononuclear cells (PBMC) can be obtained as described according to methods known in the art. Examples of such methods are described for example in: Nair, Smita et al. Current protocols in immunology vol. Chapter 7 (2012): Unit 7.32. doi: 10.1002/0471142735.im0732s99.

It is also possible to obtain a cell sample from a subject, and then to enrich it for a desired cell type. For example, PBMCs can be isolated from blood as described herein. Counter-flow centrifugation (elutriation) can be used to enrich for monocytes or dendritic cells from PBMCs. Cells can also be isolated from other cells using a variety of techniques, such as isolation and/or activation with an antibody binding to an epitope on the cell surface of the desired cell type, for example, some T-cell isolation kits use antibody conjugated beads to both activate the cells and then allow column separation with the same beads. Another method that can be used includes negative selection using antibodies to cell surface markers to selectively enrich for a specific cell type without activating the cell by receptor engagement.

In some embodiment, a population of hematopoietic stem cells is obtained from leukophoresis, bone marrow, cord blood, or peripheral blood as the source. In some embodiments, dendritic cells are obtained from pluripotent stem cells such as embryonic stem cells or induced pluripotent stem cells.

Bone marrow cells (BM cells) may be obtained from iliac crest, femora, tibiae, spine, rib or other medullary spaces. Bone marrow may be taken out of the patient and isolated through various separations and washing procedures. A known procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll® (a trademark of Pharmacia Fine Chemicals AB), and an intermediate fraction which contains the bone marrow cells is collected; and c) washing of the collected fraction from step (b) for recovery of re-transfusable bone marrow cells. Additionally, BM cells can be collected by mobilization followed by leukapheresis.

Hematopoietic stem cells (HSCs) are multipotent primitive cells that can develop into all types of blood cells, including myeloid-lineage and lymphoid-lineage cells. HSCs can be found in several organs, such as peripheral blood, bone marrow, and umbilical cord blood. HSCs are typically CD34+.

Embryonic stem cells (ESCs) come from embryos that are 3 to 5 days old. At this stage, an embryo is called a blastocyst and has about 150 cells. ESCs are pluripotent stem cells, that can divide into more stem cells or can become any type of cell in the body. Embryonic stem cells can also be obtained from ESC cell lines.

Induced pluripotent stem cells (iPSCs) are typically derived from adult skin or blood cells that have been reprogrammed back into an embryonic-like pluripotent state that enables the development of an unlimited source of any type of human cell needed for therapeutic purposes.

Dendritic cells are the sentinel antigen presenting cells of the immune system. These cells have the capacity to acquire antigenic material from their environment and to subsequently initiate vigorous immune responses, and are ideal candidates to deliver vaccines for cancer immunotherapy. Dendritic cells comprise a heterogeneous cell population with distinctive morphology and a widespread tissue distribution. DCs exhibit cell surface markers, such as CD1c+, CD14+, CD141+, CD16+, or HLA-DR+.

In particular, dendritic cells act as an antigen-presenting cell by endocytosis of exogeneous proteins which are then processed and presented as epitopes on their surface in conjunction with MHC class I and II antigens. The antigen presenting dendritic cells can be recognized by cytotoxic T cells and T-helper cells. The maturation state of the dendritic cells is important for their phagocytic/endocytic activity. Mature dendritic cells are typically the most efficient cells for loading antigens. Mature dendritic cells refer to dendritic cells which express certain cell markers including, but not limited to, CD1a, CD14, CCR7, HLA-DR, CD209, CD40, CD11c, CD86, and CD83.

In some embodiments, DCs are obtained from any tissue where they reside, including non-lymphoid tissues such as the epidermis of the skin (Langerhans cells) and lymphoid tissues such as the spleen, bone marrow, lymph nodes and thymus as well as the circulatory system including blood and peripheral blood. Because DCs occur in low numbers in the tissues in which they reside, DCs may be enriched or isolated for use. Any of a number of procedures entailing repetitive density gradient separation, positive selection, negative selection or a combination thereof may be used to obtain enriched populations of DCs. Once the DCs are obtained, they may be cultured in appropriate culture medium to expand the cell population and/or maintain the DCs in a state for optimal antigen uptake, processing and presentation.

In some embodiments, the DCs may be autologous to a subject suffering from cancer, that is, the DCs that are administered to the subject may be isolated from the same subject. In other embodiments, the DCs may be allogeneic to the subject suffering from cancer. In some embodiments, the DCs disclosed herein may be any sub-type, such as myeloid DCs, plasmacytoid DCs, interstitial DCs, lymphoid tissue resident DCs, follicular DCs, CD14+ DCs, and the like.

The cells that are used as the source of the cells for transduction with the heterologous proteins provided herein can be from any donor. In some embodiments, the donor is screened to establish that the cells isolated from the donor would be considered allogeneic to the subject to which they are administered. In some embodiments, the donor screening criteria can include donor demographics including: donor age, donor gender, donor ethnicity, donor ABO/Rh; donor BMI (weight and height), donor HLA high resolution typing.

Additionally, in some embodiments, donor samples can have a full panel testing for blood transfusion (FDA), on the source material including: CMV test; serological testing for syphilis and an antibody screen; and infectious disease panel comprising one or more of the following tests: Hepatitis B Core Antibody (Anti-HBS EIA); Hepatitis B Surface Antigen (HBsAg EIA); Hepatitis C Virus Antibody (Anti-HCV EIA); Human Immunodeficiency Virus Ab (HIV1/2 plus O); Human T-lymphotropic Virus Antibody (HTLV-I/II); HIV-1/HGV/HBV nucleic Acid testing; WNV Nucleic Acid Testing; Trypanasoma cruzi Antibody; and Zika. Thus, in some embodiments, the donor cells or the cells that are administered to the patient are derived from a donor that is free of CMV, syphilis, Hepatitis A, Hepatitis B, Hepatitis C, HIV, HTLV-I/II, West Nile Virus (WNV), T. cruzi, and/or Zika.

The samples may be evaluated for monocyte percentages and count by hematology analyzer and flow cytometry.

The samples may be collected by apheresis or by whole blood collection and subject to standard inspection. In certain instances, the collection will be by Optia® leukopheresis collection. The samples will be analyzed for viability and CD14+% and the CD14+ monocytes will be counted.

It is another option to conduct monocyte negative isolation using CliniMACS® according to standard procedures.

In some embodiments, the dendritic cells are CD34+ HSCs obtained from a variety of sources.

### Recombinant Dendritic Cells

In some embodiments, methods of developing genetically engineered/recombinant dendritic cells expressing CD40L, CXCL13, and optionally CD93, 4-1BBL, and/or IL-21, that may be used for cancer therapy are provided.

CD40L is a type II membrane protein of 35 kDa and a member of the tumor necrosis factor (TNF) gene family, is expressed on the surface of T cells upon antigen recognition. CD40L is critically involved in the activation of T cells necessary to induce an effective protective immunity against tumor self-antigens. (See, Front Immunol. 2011; 2:31, which is hereby incorporated by reference in its entirety). The full sequence information for human CD40L can be found at NCBI, Gene ID: 959.

CXCL13 is a chemokine expressed in follicular stromal cells of lymphoid organs, macrophages in the peritoneal and pleural cavities and in myeloid dendritic cells. It has been shown to bind primarily to the G-protein coupled receptor CXCR5. CXCR5 is expressed on B cells and certain subsets of T cells (including follicular helper T cells, a subset of circulating memory CD4 T cells, and other populations of T cells not fully differentiated as T-helper 1 (Th1) or T-helper 2 (Th2). CXCL13 has demonstrated physiologic roles in co-localization of B and T cells by influencing homing of auto-reactive B1 cells to Peyer's patches and other sites of inflammation, and by playing a role in recruitment of Th cells to secondary lymphoid organs for T dependent antibody production. (See, Front Immunol. 2016; 7:225, which is hereby incorporated by reference in its entirety). The full sequence information for human CXCL13 can be found at NCBI, Gene ID: 10563.

Human CD93 (hCD93) is type 1 transmembrane glycoprotein located on chromosome 20, p11.21. This protein is involved in cell-cell interaction during B cell development and phagocytosis. CD93 is expressed in myeloid lineages, hematopoietic stem cells, NK cells, platelets, microglia, and endothelial cells. The full sequence information for human CD93 can be found at NCBI, Gene ID: 22918.

4-1BBL is a protein encoded by the tumor necrosis factor ligand superfamily member 9 gene. 4-1BBL is a type 2 transmembrane glycoprotein receptor that is found on APCs (antigen presenting cells) and binds to 4-1BB (also known as CD137). The 4-1BB/4-1BBL complex belongs to the TNFR:TNF superfamily,] which is expressed on activated T lymphocytes

Interleukin-21 (IL-21) is a cytokine that has potent regulatory effects on cells of the immune system, including natural killer (NK) cells and cytotoxic T cells that can destroy virally infected or cancerous cells. This cytokine induces cell division/proliferation in its target cells.

In some embodiments, a dendritic cell comprises one or more heterologous nucleic acid molecules encoding for CD40L and/or CXCL13. The heterologous nucleic acid molecule encoding CD40L may be from any source, such as human, mouse, rat, or pig, or any other mammal. Human CD40L nucleic acid sequence is disclosed as SEQ ID NO:1. Similarly, the heterologous nucleic acid molecule encoding CXCL13 may be from any source, such as human, mouse, rat, or pig, or any other mammal. The human CXCL13 nucleic acid sequence is disclosed as SEQ ID NO:2.

In some embodiments, the dendritic cells may further comprise heterologous nucleic acid molecule encoding CD93, 4-1BBL, and/or IL-21. The heterologous nucleic acid molecule encoding CD93 may be from any source, such as human, mouse, rat, or pig, or any other mammal. The human CD93 nucleic acid sequence is disclosed as SEQ ID NO:3. The human 4-1BBL nucleic acid sequence is disclosed as SEQ ID NO:7. The human IL-21 nucleic acid sequence is disclosed as SEQ ID NO:8.

The nucleic acid sequences encoding the proteins, however, can be other sequences due to the degenerate nature of the genetic code. These nucleic acid sequences are non-limiting examples and other can be used.

The corresponding human amino acid sequences are as follows: human CD40L: SEQ ID NO: 4; human CXCL13: SEQ ID NO:5; human CD93: SEQ ID NO: 6, human 4-1-BBL: SEQ ID NO: 9, and human IL-21: SEQ ID NO:10. In some embodiments, the protein comprises a conservative substitution.

The following sequences can be used as reference throughout the present application as appropriate:

In some embodiments, human CD40L is encoded by:
(SEQ ID NO: 1)
ATGATCGAAACATACAACCAAACTTCTCCCCGATCTGCGGCCACTGGACTGCCCATCAGCATGAAAATTT
TTATGTATTTACTTACTGTTTTTCTTATCACCCAGATGATTGGGTCAGCACTTTTTGCTGTGTATCTTCA
TAGAAGGTTGGACAAGATAGAAGATGAAAGGAATCTTCATGAAGATTTTGTATTCATGAAAACGATACAG
AGATGCAACACAGGAGAAAGATCCTTATCCTTACTGAACTGTGAGGAGATTAAAAGCCAGTTTGAAGGCT
TTGTGAAGGATATAATGTTAAACAAAGAGGAGACGAAGAAAGAAAACAGCTTTGAAATGCAAAAAGGTGA
TCAGAATCCTCAAATTGCGGCACATGTCATAAGTGAGGCCAGCAGTAAAACAACATCTGTGTTACAGTGG
GCTGAAAAAGGATACTACACCATGAGCAACAACTTGGTAACCCTGGAAAATGGGAAACAGCTGACCGTTA
AAAGACAAGGACTCTATTATATCTATGCCCAAGTCACCTTCTGTTCCAATCGGGAAGCTTCGAGTCAAGC
TCCATTTATAGCCAGCCTCTGCCTAAAGTCCCCCGGTAGATTCGAGAGAATCTTACTCAGAGCTGCAAAT
ACCCACAGTTCCGCCAAACCTTGCGGGCAACAATCCATTCACTTGGGAGGAGTATTTGAATTGCAACCAG
GTGCTTCGGTGTTTGTCAATGTGACTGATCCAAGCCAAGTGAGCCATGGCACTGGCTTCACGTCCTTTGG
CTTACTCAAACTCTGA
In some embodiments, human CXCL13 is encoded by:
(SEQ ID NO: 2)
ATGAAGTTCATCTCGACATCTCTGCTTCTCATGCTGCTGGTCAGCAGCCTCTCTCCAGTCCAAGGTGTTC
TGGAGGTCTATTACACAAGCTTGAGGTGTAGATGTGTCCAAGAGAGCTCAGTCTTTATCCCTAGACGCTT
CATTGATCGAATTCAAATCTTGCCCCGTGGGAATGGTTGTCCAAGAAAAGAAATCATAGTCTGGAAGAAG
AACAAGTCAATTGTGTGTGTGGACCCTCAAGCTGAATGGATACAAAGAATGATGGAAGTATTGAGAAAAA
GAAGTTCTTCAACTCTACCAGTTCCAGTGTTTAAGAGAAAGATTCCC
In some embodiments, human CD93 is encoded by:
(SEQ ID NO: 3)
ATGGCCACCTCCATGGGCCTGCTGCTGCTGCTGCTGCTGCTCCTGACCCAGCCCGGGGGGGGACGGGAG
CTGACACGGAGGCGGTGGTCTGCGTGGGGACCGCCTGCTACACGGCCCACTCGGGCAAGCTGAGCGCTGC
CGAGGCCCAGAACCACTGCAACCAGAACGGGGGCAACCTGGCCACTGTGAAGAGCAAGGAGGAGGCCCAG
CACGTCCAGCGAGTACTGGCCCAGCTCCTGAGGCGGGAGGCAGCCCTGACGGCGAGGATGAGCAAGTTCT
GGATTGGGCTCCAGCGAGAGAAGGGCAAGTGCCTGGACCCTAGTCTGCCGCTGAAGGGCTTCAGCTGGGT
GGGCGGGGGGGAGGACACGCCTTACTCTAACTGGCACAAGGAGCTCCGGAACTCGTGCATCTCCAAGCGC
TGTGTGTCTCTGCTGCTGGACCTGTCCCAGCCGCTCCTTCCCAGCCGCCTCCCCAAGTGGTCTGAGGGCC
CCTGTGGGAGCCCAGGCTCCCCCGGAAGTAACATTGAGGGCTTCGTGTGCAAGTTCAGCTTCAAAGGCAT
GTGCCGGCCTCTGGCCCTGGGGGGCCCAGGTCAGGTGACCTACACCACCCCCTTCCAGACCACCAGTTCC
TCCTTGGAGGCTGTGCCCTTTGCCTCTGCGGCCAATGTAGCCTGTGGGGAAGGTGACAAGGACGAGACTC
AGAGTCATTATTTCCTGTGCAAGGAGAAGGCCCCCGATGTGTTCGACTGGGGCAGCTCGGGCCCCCTCTG
TGTCAGCCCCAAGTATGGCTGCAACTTCAACAATGGGGGCTGCCACCAGGACTGCTTTGAAGGGGGGGAT
GGCTCCTTCCTCTGCGGCTGCCGACCAGGATTCCGGCTGCTGGATGACCTGGTGACCTGTGCCTCTCGAA
ACCCTTGCAGCTCCAGCCCATGTCGTGGGGGGGCCACGTGCGTCCTGGGACCCCATGGGAAAAACTACAC
GTGCCGCTGCCCCCAAGGGTACCAGCTGGACTCGAGTCAGCTGGACTGTGTGGACGTGGATGAATGCCAG
GACTCCCCCTGTGCCCAGGAGTGTGTCAACACCCCTGGGGGCTTCCGCTGCGAATGCTGGGTTGGCTATG
AGCCGGGCGGTCCTGGAGAGGGGGCCTGTCAGGATGTGGATGAGTGTGCTCTGGGTCGCTCGCCTTGCGC
CCAGGGCTGCACCAACACAGATGGCTCATTTCACTGCTCCTGTGAGGAGGGCTACGTCCTGGCCGGGGAG
GACGGGACTCAGTGCCAGGACGTGGATGAGTGTGTGGGCCCGGGGGGCCCCCTCTGCGACAGCTTGTGCT
TCAACACACAAGGGTCCTTCCACTGTGGCTGCCTGCCAGGCTGGGTGCTGGCCCCAAATGGGGTCTCTTG
CACCATGGGGCCTGTGTCTCTGGGACCACCATCTGGGCCCCCCGATGAGGAGGACAAAGGAGAGAAAGAA
GGGAGCACCGTGCCCCGTGCTGCAACAGCCAGTCCCACAAGGGGCCCCGAGGGCACCCCCAAGGCTACAC
CCACCACAAGTAGACCTTCGCTGTCATCTGACGCCCCCATCACATCTGCCCCACTCAAGATGCTGGCCCC
CAGTGGGTCCCCAGGCGTCTGGAGGGAGCCCAGCATCCATCACGCCACAGCTGCCTCTGGCCCCCAGGAG
CCTGCAGGTGGGGACTCCTCCGTGGCCACACAAAACAACGATGGCACTGACGGGCAAAAGCTGCTTTTAT
TCTACATCCTAGGCACCGTGGTGGCCATCCTACTCCTGCTGGCCCTGGCTCTGGGGCTACTGGTCTATCG
CAAGCGGAGAGCGAAGAGGGAGGAGAAGAAGGAGAAGAAGCCCCAGAATGCGGCAGACAGTTACTCCTGG
GTTCCAGAGCGAGCTGAGAGCAGGGCCATGGAGAACCAGTACAGTCCGACACCTGGGACAGACTGC
In some embodiments, human 4-1BBL (also known as TNFSF9; CD137L; TNLG5A;
4-1BB-L) is encoded by:
(SEQ ID NO: 7; NCBI NM_003811.4)(ns))
ATGGAATACGCCTCTGACGCTTCACTGGACCCCGAAGCCCCGTGGCCTCCCGCGCCCCGCGCTCGCGCCT
GCCGCGTACTGCCTTGGGCCCTGGTCGCGGGGCTGCTGCTGCTGCTGCTGCTCGCTGCCGCCTGCGCCGT
CTTCCTCGCCTGCCCCTGGGCCGTGTCCGGGGCTCGCGCCTCGCCCGGCTCCGCGGCCAGCCCGAGACTC
CGCGAGGGTCCCGAGCTTTCGCCCGACGATCCCGCCGGCCTCTTGGACCTGCGGCAGGGCATGTTTGCGC
AGCTGGTGGCCCAAAATGTTCTGCTGATCGATGGGCCCCTGAGCTGGTACAGTGACCCAGGCCTGGCAGG
CGTGTCCCTGACGGGGGGCCTGAGCTACAAAGAGGACACGAAGGAGCTGGTGGTGGCCAAGGCTGGAGTC
TACTATGTCTTCTTTCAACTAGAGCTGCGGCGCGTGGTGGCCGGCGAGGGCTCAGGCTCCGTTTCACTTG
CGCTGCACCTGCAGCCACTGCGCTCTGCTGCTGGGGCCGCCGCCCTGGCTTTGACCGTGGACCTGCCACC
CGCCTCCTCCGAGGCTCGGAACTCGGCCTTCGGTTTCCAGGGCCGCTTGCTGCACCTGAGTGCCGGCCAG
CGCCTGGGCGTCCATCTTCACACTGAGGCCAGGGCACGCCATGCCTGGCAGCTTACCCAGGGCGCCACAG
TCTTGGGACTCTTCCGGGTGACCCCCGAAATCCCAGCCGGACTCCCTTCACCGAGGTCGGAA
In some embodiments, human IL-21 is encoded by:
(SEQ ID NO: 8; NCBI NM_021803.4)
ATGAGATCCAGTCCTGGCAACATGGAGAGGATTGTCATCTGTCTGATGGTCATCTTCTTGGGGACACTGG
TCCACAAATCAAGCTCCCAAGGTCAAGATCGCCACATGATTAGAATGCGTCAACTTATAGATATTGTTGA
TCAGCTGAAAAATTATGTGAATGACTTGGTCCCTGAATTTCTGCCAGCTCCAGAAGATGTAGAGACAAAC
TGTGAGTGGTCAGCTTTTTCCTGCTTTCAGAAGGCCCAACTAAAGTCAGCAAATACAGGAAACAATGAAA
GGATAATCAATGTATCAATTAAAAAGCTGAAGAGGAAACCACCTTCCACAAATGCAGGGAGAAGACAGAA
ACACAGACTAACATGCCCTTCATGTGATTCTTATGAGAAAAAACCACCCAAAGAATTCCTAGAAAGATTC
AAATCACTTCTCCAAAAGATGATTCATCAGCATCTGTCCTCTAGAACACACGGAAGTGAAGATTCCTGA
In some embodiments, human CD40L comprises an amino acid sequence of:
(SEQ ID NO: 4)
MIETYNQTSPRSAATGLPISMKIFMYLLTVFLITQMIGSALFAVYLHRRLDKIEDERNLHEDFVFMKTIQ
RCNTGERSLSLLNCEEIKSQFEGFVKDIMLNKEETKKENSFEMQKGDQNPQIAAHVISEASSKTTSVLQW
AEKGYYTMSNNLVTLENGKQLTVKRQGLYYIYAQVTFCSNREASSQAPFIASLCLKSPGRFERILLRAAN
THSSAKPCGQQSIHLGGVFELQPGASVFVNVTDPSQVSHGTGFTSFGLLKL*
In some embodiments, human CXCL13 comprises an amino acid sequence of:
(SEQ ID NO: 5)
MKFISTSLLLMLLVSSLSPVQGVLEVYYTSLRCRCVQESSVFIPRRFIDRIQILPRGNGCPRKEIIVWKK
NKSIVCVDPQAEWIQRMMEVLRKRSSSTLPVPVFKRKIP
In some embodiments, human CD93 comprises an amino acid sequence of:
(SEQ ID NO: 6)
MATSMGLLLLLLLLLTQPGAGTGADTEAVVCVGTACYTAHSGKLSAAEAQNHCNONGGNLATVKSKEEAQ
HVORVLAQLLRREAALTARMSKFWIGLQREKGKCLDPSLPLKGFSWVGGGEDTPYSNWHKELRNSCISKR
CVSLLLDLSQPLLPSRLPKWSEGPCGSPGSPGSNIEGFVCKFSFKGMCRPLALGGPGQVTYTTPFQTTSS
SLEAVPFASAANVACGEGDKDETQSHYFLCKEKAPDVFDWGSSGPLCVSPKYGCNENNGGCHQDCFEGGD
GSFLCGCRPGFRLLDDLVTCASRNPCSSSPCRGGATCVLGPHGKNYTCRCPQGYQLDSSQLDCVDVDECQ
DSPCAQECVNTPGGFRCECWVGYEPGGPGEGACQDVDECALGRSPCAQGCTNTDGSFHCSCEEGYVLAGE
DGTQCQDVDECVGPGGPLCDSLCFNTQGSFHCGCLPGWVLAPNGVSCTMGPVSLGPPSGPPDEEDKGEKE
GSTVPRAATASPTRGPEGTPKATPTTSRPSLSSDAPITSAPLKMLAPSGSPGVWREPSIHHATAASGPQE
PAGGDSSVATQNNDGTDGQKLLLFYILGTVVAILLLLALALGLLVYRKRRAKREEKKEKKPQNAADSYSW
VPERAESRAMENQYSPTPGTDC
In some embodiments, human 4-1BBL comprises an amino acid sequence of:
(SEQ ID NO: 9)
MEYASDASLDPEAPWPPAPRARACRVLPWALVAGLLLLLLLAAACAVFLACPWAVSGARASPGSAASPRL
REGPELSPDDPAGLLDLRQGMFAQLVAQNVLLIDGPLSWYSDPGLAGVSLTGGLSYKEDTKELVVAKAGV
YYVFFQLELRRVVAGEGSGSVSLALHLQPLRSAAGAAALALTVDLPPASSEARNSAFGFQGRLLHLSAGQ
RLGVHLHTEARARHAWQLTQGATVLGLFRVTPEIPAGLPSPRSE
In some embodiments, human IL-21 comprises an amino acid sequence of:
(SEQ ID NO: 10)
MRSSPGNMERIVICLMVIFLGTLVHKSSSQGQDRHMIRMRQLIDIVDQLKNYVNDLVPEFLPAPEDVETN
CEWSAFSCFQKAQLKSANTGNNERIINVSIKKLKRKPPSTNAGRRQKHRLTCPSCDSYEKKPPKEFLERF
KSLLQKMIHQHLSSRTHGSEDS*

In some embodiments, protein is at least, or about, 85, 88, 90, 91, 92, 93, 94, 95, 96, 97, 98, 99% homologous to the sequences provided herein. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Also included are polypeptides which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the present disclosure are not limited to products of any of the specific exemplary processes listed herein.

In addition to substantially full length polypeptides, the present disclosure provides for biologically active fragments of the polypeptides.

The heterologous nucleic acid molecules can be introduced into dendritic cells by any known recombinant techniques known in the art, such as liposomal transfection, chemical transfection, transgenic DNA recombination, viral infection, transposon insertion, jumping gene insertion, micro-injection, electroporation, gene-gun penetration, and a combination thereof. In some embodiments, a recombinant adenoviral vector, a recombinant adeno-associated vector, a recombinant retroviral vector, a recombinant lentiviral vector, or a combination thereof, may be used to introduce the heterologous nucleic acid molecules.

In some embodiments, methods to express desired genes CD40L, CXCL13, CD93, 4-1BBL, and IL-21 may be used including delivering CRISPR/Cas system, TALENs, zinc-finger nucleases. For example, TALENs and CRISPR/Cas systems may be used to insert specific transcriptional regulatory elements upstream and downstream of a native gene. TALENs and CRISPR/Cas9 are capable of generating single or double stranded DNA breaks at specific loci. This stimulates homology directed repair (HDR) from an exogenous template allowing for precise insertion of transcriptional regulatory elements to activate the native genes. CRISPR/Cas nucleases may be delivered by using any gene delivery vectors, such as adenoviral vector, adeno-associated vector, retroviral vector, lentiviral vector, or a combination thereof. Additionally, the DCs may be electroporated with mRNAs encoding the desired genes.

In some embodiments, the genetically engineered DCs disclosed herein may be cultured in conventional nutrient media under ambient conditions, such as temperature, pH, and the like, and are apparent to those skilled in the art. The DCs may be cultured in, automated, closed, or open systems.

### Antigen Activation of DCs

In some embodiments, the DCs overexpressing CD40L, CXCL13, and optionally CD93, 4-1BBL, and/or IL-21 may be activated or pulsed by an antigen. In some embodiments, the antigen may be a tumor antigen or a viral antigen. Non-limiting examples of tumor antigens include antigens expressed by a cancer cell line or a cancer cell lysate from a primary or metastatic tumor. In some embodiments, the tumor antigen is expressed by a colorectal cancer cell, a breast cancer cell, an ovarian cancer cell, a pancreatic cancer cell, a head and neck cancer cell, a bladder cancer cell, a liver cancer cell, a renal cancer cell, a melanoma cell, a gastrointestinal cancer cell, a prostate cancer cell, a small cell lung cancer cell, a non-small cell lung cancer cell, a sarcoma cell, a glioblastoma cell, a T- or B-cell lymphoma cell, an endometrial cancer cell, or a cervical cancer cell.

In some embodiments, the antigen may be a tumor (cancer) cell lysate, and the tumor cell lysate may be allogeneic or autologous to the dendritic cell. In some embodiments, the tumor cell lysate my be allogeneic or autologous to the recipient of the DC therapy. In some embodiments, the antigen source may be peptides isolated from a lysate or a recombinant antigen, or synthesized peptides.

Numerous methods of pulsing or activating dendritic cells with antigen are known in the art. In some embodiments, the antigen may be added to cultured dendritic cells under conditions promoting viability of the cells, and the cells are then allowed sufficient time to take up and process the antigen, and express antigen peptides on the cell surface in association with either Class I or Class II MHC, a period of about 4 hours to about 24 hours. Dendritic cells may also be exposed to antigen by transfecting them with DNA encoding the antigen. The DNA is expressed, and the antigen is presumably processed via the cytosolic/Class I pathway.

In some embodiments, isolated peptides may be used to pulse or activate DCs. Peptide is pulsed by any of a variety of methods, including incubation of the peptide with the dendritic cell, incubation of a protein comprising the peptide with DC, transduction of DC (or the progenitor expanded monocyte population) with a gene encoding the peptide (or a protein comprising the peptide), or the like. Typical antigens for use as peptides are derived from those expressed in a target cell such as a transformed cell, a cancer cell, a bacterial cell, a parasitically infected cell or a virally infected cell, or the like. Examples of antigens include, but are not limited to, carbohydrates, such as mucin, tumor antigens, peptides derived from a protein selected from the group consisting of HIV Gag, HIV Env, HER-2, MART-1, gp-100, PSA, HBVc, HBVs, HPV E6, HPV E7, tyrosinase, MAGE-1, trp-1, mycobacterial antigens, and CEA, as well as many others. Tumor antigens suitable for presentation include, but are not limited to, c-erb-B-2/HER2/neu, PEM/MUC-1, Int-2, Hst, BRCA-1, BRCA-2, truncated EGFRvlll, CEA, p53, ras, RK, Myc, Myb, OB-1, OB-2, BCR/ABL, GIP, GSP, RET, ROS, FIS, SRC, TRC, WTI, DCC, NF1, FAP, MEN-1, ERB-B1, MAGE-antigens, and idiotypic immunoglobulins (e.g., from a B cell of a non-Hodgkin's lymphoma patient). The antigen presenting activity of dendritic cells may be enhanced by co-culture with certain cytokines, such as TNF-α or IL-1a or IL-1B.

The activated dendritic cells disclosed herein may be present in a composition comprising physiologically acceptable carriers, excipients, adjuvants, or diluents. Neutral buffered saline or saline mixed with serum albumin are exemplary appropriate diluents. Suitable carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In some embodiments, the composition may comprise a tumor antigen or a viral antigen. In some embodiments, the composition is free of any heterologous antigen. In some embodiments, the composition comprises a population of at least about 10⁵ dendritic cells/mL, at least about 10⁶ cells/mL, at least about 10⁷ cells/mL, or more.

In some embodiments, the activated DCs may be frozen (cryopreserved) before administration into a subject. In some embodiments, the activated DCs may be frozen by suspending the cells in media containing at least 30% human-derived serum and/or plasma, and lowering the temperature of the suspension to at least −80° C., thereby freezing the DCs. In some embodiments, the freezing media is approximately 30% human-derived serum and/or plasma and approximately 10% of an agent that prevents ice crystal formation during freezing, e.g., DMSO. In a further embodiment, the DC suspension is maintained at −80° C. for at least 24 hours and then transferred to liquid nitrogen for the duration of the storage. In a further embodiment, the DC suspension is thawed at a temperature in the range of 34° to 41° C. In some embodiments, the cryopreservation buffer contains Plasma-Lyte A (isotonic solution wherein each 100 mL contains 526 mg NaCl, 502 mg sodium gluconate (C₆H₁₁NaO₇), 368 mg sodium acetate trihydrate (C₂H₃NaO₂·3H₂O), 37 mg KCl, and 30 mg MgCl₂·6H₂O), human serum albumin, and CryoStor® CS10 (dimethyl sulfoxide and saccharose).

Also disclosed herein are methods to develop immature DCs from monocytes or HSCs, maturing the immature DCs to mature DCs, and activating the mature DCs with allogenic or autologous tumor cell lysates. In some embodiments, the method includes: (a) isolating monocytes or HSCs from a subject or obtaining a source of HSCs; (b) differentiating the monocytes or HSCs into immature dendritic cells in vitro, (c) maturing the immature dendritic cells in vitro into mature dendritic cells, and (d) overexpressing one or more genes selected from CD40L, CXCL13, CD93, 4-1BBL, and IL-21 in the immature or mature dendritic cells. In some embodiments, the monocytes are CD14+ monocytes. In some embodiments, the HSCs are CD34+ HSCs. The different combinations of the heterologously expressed proteins are also described herein.

In some embodiments, the monocytes are obtained from a variety of sources such as, but not limited to, leukapheresis of peripheral blood mononuclear cells from a patient, followed by elutriation of the isolated peripheral blood to provide isolated monocytes. In some embodiments, the HSCs are obtained from a variety of sources such as, but not limited to, peripheral blood, cord blood, bone marrow, and leukophoresis.

The dendritic cells may be genetically manipulated to express any one or combination of the heterologous CD40L, CXCL13, and optionally CD93, 4-1BBL, and/or IL-21 proteins by techniques disclosed herein, such as liposomal transfection, chemical transfection, transgenic DNA recombination, viral infection, transposon insertion, jumping gene insertion, micro-injection, electroporation, gene-gun penetration, and a combination thereof. In some embodiments, a recombinant adenoviral vector, a recombinant adeno-associated vector, a recombinant retroviral vector, a recombinant lentiviral vector, RNA electroporation, or a combination thereof, may be used. Methods such as delivering CRISPR/Cas system, TALENs, and zinc-finger nucleases may also be used to overexpress any one or combination of the heterologous proteins CD40L, CXCL13, and optionally CD93, 4-1BBL, and/or IL-21. In some embodiments, immature DCs are genetically-modified to express one or more heterologous proteins. In some embodiments, mature DCs are genetically-modified to express one or more heterologous proteins.

In some embodiments, the monocytes or HSCs are grown in the presence of IL-3, causing the monocytes or HSCs to proliferate, yielding an expanded population of cells for production of DCs. The expanded population of cells is differentiated into immature dendritic cells, e.g., by culturing the expanded population of cells with GM-CSF and IL-4 (to produce baseline or Type I DCs) and, optionally one or more of TNF-α, IL-1β, IL-6, IFN-α, IFN-γ, and PGE2. The immature dendritic cells are then differentiated into mature dendritic cells e.g., by culturing the immature dendritic cells in a AIM V™ Media containing human serum and one or more of, IL-4, GMCF, TNF-α, PGE2, IL-6, IL-1b, IL-12, and IL15.

In some embodiments, the recombinant allogeneic mature dendritic cells may be activated or pulsed by an antigen. In some embodiments, the antigen is a tumor antigen or a viral antigen. In some embodiments, the antigen may be a tumor cell lysate, and the tumor cell lysate may be allogenic or autologous to the immature dendritic cell. In some embodiments, the cells are not activated or pulsed by an antigen.

In some embodiments, the activated DCs disclosed herein may be present in a composition comprising physiologically acceptable carriers, excipients, adjuvants, or diluents. Neutral buffered saline or saline mixed with serum albumin are exemplary appropriate diluents. In some embodiments, the activated immature DCs may be frozen before administration into a subject. The cells can be thawed prior to being administered to the subject.

### Treatment of Cancer

Also disclosed herein are methods of treating cancer in a subject. In some embodiments, a method of treating cancer in a subject comprises administering to the subject a composition comprising a recombinant dendritic cell, wherein the dendritic cell heterologously expresses one or more proteins selected from CD40L, CXCL13, and optionally CD93, 4-1BBL, and/or IL-21. As described herein, the cells can be allogeneic to the subject. The cells, in some embodiments, can be autologous to the subject.

In some embodiments, a method of eliciting immune response in a subject suffering from cancer comprises administering to the subject a composition comprising a dendritic cell, wherein the dendritic cell heterologously expresses one or more proteins selected from CD40L, CXCL13, and optionally CD93, 4-1BBL, and/or IL-21. In some embodiments, the DCs heterologously express CD40L and CXCL13. In some embodiments, the DCs heterologously express CD40L, CXCL13, and CD93. In some embodiments, the DCs heterologously express CD40L and CD93. In some embodiments, the DCs heterologously express CXCL13 and CD93. In some embodiments, the DCs do not heterologously express CD93. In some embodiments, the DCs heterologously express 4-1BBL. In some embodiments, the DCs heterologously express IL-21. In some embodiments, the DCs do not heterologously express 4-1BBL. In some embodiments, the DCs do not heterologously express IL-21.

In some embodiments, provided herein are methods of providing a cancer vaccine to a subject suffering from cancer comprises administering to the subject a composition comprising a dendritic cell, wherein the dendritic cell heterologously express one or more proteins selected from CD40L, CXCL13, and optionally CD93, 4-1BBL, and/or IL-21. In some embodiments, the DCs heterologously express CD40L and CXCL13. In some embodiments, the DCs heterologously express CD40L, CXCL13, and CD93. In some embodiments, the DCs heterologously express CD40L and CD93. In some embodiments, the DCs heterologously express CXCL13 and CD93. In some embodiments, the DCs do not heterologously express CD93. In some embodiments, the DCs heterologously express 4-1BBL. In some embodiments, the DCs heterologously express IL-21. In some embodiments, the DCs do not heterologously express 4-1BBL. In some embodiments, the DCs do not heterologously express IL-21.

In some embodiments, the subject is suffering from a cancer selected from the group consisting of colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, merkel cell carcinoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, acute myelocytic leukemia, chronic leukemia, polycythemia vera, lymphoma, multiple myeloma, Waldenstrom's macroglobulinemia, heavy chain disease, and combinations thereof.

In additional embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases.

In additional embodiments, the method further comprises at a time-frame of from 4-14 days following administration of the recombinant dendritic cells, administering the patient an immune check-point inhibitor, including any one or combination of two check point inhibitors, including an inhibitor of PD-1 or PD-L1 (B7-H1), such as an anti-PD-1 antibody, including nivolumab (Opdivo®, Bristol-Myers Squibb), pembrolizumab/lambrolizumab, also known as MK-3475 (Keytruda®, Merck), pidilizumab (Curetech), AMP-224 (Amplimmune), or an anti-PD-L1 antibody, including MPDL3280A (Roche), MDX-1105 (Bristol-Myers Squibb), MEDI-4736 (AstraZeneca) and MSB-0010718 C (Merck), an antagonist of CTLA-4, such as an anti-CTLA-4 antibody including anti-CTLA4 antibody Yervoy® (ipilimumab, Bristol-Myers Squibb), tremelimumab (Pfizer), ticilimumab (AstraZeneca), or AMGP-224 (Glaxo SmithKline), or a tumor specific antibody trastuzumab (Herceptin®) for breast cancer, rituximab (Rituxan®) for lymphoma, or cetuximab (Erbitux®).

In additional embodiments, the treatment, administration, or increasing the immune response is repeated periodically for time frames, for as long as the patient exhibits improvement or stable/non-progressing disease.

In additional embodiments, the treatment, administration, or increasing the immune response is repeated periodically for time frames of from once every 5 days, once every week, once every 14 days, once every 21 days, to once a month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment, for as long as the patient exhibits improvement or stable/non-progressing disease.

In some embodiments, the recombinant dendritic cells (heterologously CD40L, CXCL13, and/or CD93, 4-1BBL, and IL-21) administered to the subject are not activated or pulsed with an antigen prior to administration. In some embodiments, the dendritic cells may be administered in a composition comprising adjuvants, cytokines, and interleukins that may help in eliciting immune response. In some embodiments, the DCs heterologously express CD40L and CXCL13. In some embodiments, the DCs heterologously express CD40L, CXCL13, and CD93. In some embodiments, the DCs do not heterologously express CD93. In some embodiments, the DCs heterologously express 4-1BBL. In some embodiments, the DCs heterologously express IL-21. In some embodiments, the DCs do not heterologously express 4-1BBL. In some embodiments, the DCs do not heterologously express IL-21.

In some embodiments, the method further comprises: (a) obtaining a protein expression profile of a resected tumor or biopsy sample from the subject; (b) comparing the protein expression profile of the resected tumor or biopsy sample to the protein expression profile of a cancer cell lysate; and (c) if at least one marker in the protein expression profile of the resected tumor or biopsy sample match with the protein expression profile of the cancer cell lysate, then co-culturing the dendritic cell overexpressing CD40L, CXCL13, and optionally CD93, 4-1BBL, and IL-21 with the cancer cell lysate to activate the dendritic cell, and administering to the subject a composition comprising the activated dendritic cell; and (d) if at least one marker in the protein expression profile of the resected tumor or biopsy sample do not match with the protein expression profile of the cancer cell lysate, then co-culturing the dendritic cell overexpressing CD40L, CXCL13, and optionally CD93, 4-1BBL, and IL-21 with the tumor or biopsy sample to activate the dendritic cell, and administering to the subject a composition comprising the activated dendritic cell.

In some embodiments, the methods provided for herein further comprise, screening a protein expression profile of a resected tumor or biopsy sample from the subject to cross-match a protein expression profile of an allogeneic tumor lysate prior to administration; and administering an allogeneic tumor lysate activated dendritic cell or activated dendritic cell composition if at least one fragment of the protein expression profile of the resected tumor or biopsy sample cross-match the protein expression profile of the allogeneic tumor lysate. As used herein, the term “cross-match” refers to comparing the protein expression profile of one sample against another, such as the tumor lysate as compared to the biopsy or resected tumor sample. If a protein fragment is found in both the lysate and the sample it is said to match.

In some embodiments, the protein expression profile is measured or compared by well-known techniques in the art, such as protein arrays, proteomics, mass spectroscopy (MALDI-MS), and the like. In some embodiments, gene expression profile may be used in place of protein expression profile for comparison. Gene expression profile can be obtained using well-known techniques in the art, such as gene arrays, microarrays, RT-PCR, and the like.

In some embodiments, the route of administration is via, intratumoral, peritumoral, intradermal, subcutaneous, intramuscular, intraperitoneal injection. The compositions are administered to stimulate an immune response, and can be given by bolus injection, continuous infusion, sustained release from implants, or other suitable technique.

For the purpose of illustration only, the method can be practiced by obtaining and saving blood samples from the subject prior to infusion for subsequent analysis and comparison. Generally at least about 10⁶ to 10⁸ cells in total, in some embodiments between 10⁷ and 10⁸ cells in total, and in some further embodiments, between about 4×10⁷ and 8×10⁷ cells are injected intradermally, subcutaneous, or infused intravenously or intraperitoneally into a 70 kg patient over roughly 60-120 minutes.

In some embodiments, the total number of cells are administered in multiple injections over a period of time. In some embodiments, the total number of cells are administered in up to six intradermal injections. A treatment regiment can have multiple courses, each course can have multiple sets of injections. Dosing dependent on number of cells to be administered. Each course repeats every 1-4 weeks for up to six courses.

### Kits

Additionally, certain components or embodiments of these recombinant dendritic cell compositions can be provided in a kit. For example, any of the recombinant dendritic cell compositions, as well as the autologous or other tumor cell lysate compositions can be provided frozen and packaged as a kit, alone or along with separate containers of any of the other agents from the pre-conditioning or post-conditioning steps, and optional instructions for use.

Some embodiments are also directed to any of the aforementioned cellular compositions in a kit. In some embodiments, the kit may comprise ampoules, disposable syringes, capsules, vials, tubes, or the like. In some embodiments, the kit may comprise a single dose container or multiple dose containers comprising the topical formulation of embodiments herein. In some embodiments, each dose container may contain one or more unit doses. In some embodiments, the kit may include an applicator. In some embodiments, the kits include all components needed for the stages of conditioning/treatment. In some embodiments, the cellular compositions may have preservatives or be preservative-free (for example, in a single-use container). In some embodiments, the recombinant dendritic cell compositions expressing any one or more of CD40L, CXCL13, CD93, 4-1BBL, or IL-21 may be prepared and frozen in an immature stage, suitable for shipping to a hospital or treatment center. In some embodiments, the antigens for loading, either autologous or f tumor cell lysate derived from a cell line can be prepared and frozen separately from the recombinant dendritic cell compositions, using standard methods, such that these compositions can be shipped to a hospital or treatment center for further processing and administration to the patient. In yet further embodiments, the recombinant dendritic cell compositions expressing any one or more of CD40L, CXCL13, CD93, 4-1BBL, or IL-21 may be prepared and mixed with the desired autologous or cell lysate derived from a cell line or primary or metastatic tumor to facilitate loading of the recombinant dendritic cells with the tumor antigens, after which this mixture is frozen, such that these compositions can be shipped to a hospital or treatment center for further processing and administration to the patient.

In some embodiments, the cell lysate is from a tumor lysate, such as a pancreas or liver tumor lysate. In some embodiments, the cell lysate is from a tumor cell line, such as a pancreas or liver tumor cell line. In some embodiments, the pancreas cell line is MIA PaCa-2 (MP2).

In some embodiments, for non-resectable and non-biopsy patients, CD40L+CXCL13 (and optionally CD93, 4-1BBL, or IL-21) recombinant alloDC can be administered as an immune adjuvant without antigen pulsing or activation.

### Post Isolation Purity and Cell Count

In some embodiments, purified monocytes, HSCs, immature DCs, or mature DCs are electroporated with mRNAs encoding CD40L and CXCL13 (and for certain embodiments CD40L, CXCL13, and CD93. and optionally 4-1BBL or IL-21).

In some embodiments, purified monocyte, HSCs, immature DCs, or mature DCs are transduced with a lentiviral vector comprising CD40L and CXCL13 (and for certain embodiments CD40L, CXCL13, and CD93, and optionally 4-1BBL or IL-21).

After exposure to the viral vector, the positively transduced cells can be selected. For example, the cells can be selected with one or more of the following positive or negative markers: Linage negative (CD3−, CD56−, CD19−, CD66b−), CD45+, CD14+, CD40L+, CXCL13+, CD1c+, CD11b+, CD11c+, HLA-DR+, CD86+, CD80low, CD83−, CD16Low, CD33+, CD163−, CD206+, or CD209.

In certain instances, the transduction efficiency of cells will be evaluated, prior to positive sorting so that yield can be calculated. Transduction efficiency is required/useful to determine the expected yield of CD40L+CXCL13+ or CD40L+CXCL13+CD93+ cells.

As provided herein, the cells can be differentiated into immature dendritic cells in vitro. In some embodiments, the cells (transduced or non-transduced) are centrifuge to isolate the cells. The cells can be, for example, centrifuged at 400×g for 10 minutes at room temperature (RT). The centrifuged cells can be separated from their supernatant. The centrifuged cells can be resuspended with X-VIVO™ 15 medium, recombinant human GM-CSF, such as (1000 Unit/ml) and IL-4 (1000 Unit/ml) in cell culture flask, and the cells can be allowed to differentiate the monocytes in incubator for at least or about 1, 2, 3, 4, 5, 6, 7, 8, 9, or 10 days. In some embodiments, the cells are allowed to differentiate for about 4 to about 8 days.

In some embodiments, the differentiated cells, can then be collected from the cell culture media and centrifuged. For example, in some embodiments, the cell culture media is collected from the culture flasks into centrifuge tubes. PBS can be placed into cell culture flasks to cover the surface of the flasks. The cell culture flask can be incubated with PBS at 37° C., 5% CO₂ incubator for about 30 minutes. In some embodiments, the contents of the flasks are collected into the centrifuge tubes by tapping the flasks after incubation. The flasks can be rinsed and collected into centrifugation tubes. In some embodiments, the collected cells can be centrifuged, for example, at 400×g for 10 minutes at room temperature (RT) with low break. After centrifugation, the cells are separated from their supernatant and the cells are isolated. The cells can be re-suspend and analyzed for viability.

In some embodiments, the samples are analyzed for count and flow cytometry identification of related biomarkers. They can be analyzed by or can be selected with one or more of the following positive or negative markers: Linage negative (CD3−, CD56−, CD19−, CD66b−), CD45+, CD14+, CD40L+, CXCL13+, CD1c+, CD11b+, CD11c+, HLA-DR+, CD86+, CD80low, CD83−, CD16Low, CD33+, CD163−, CD206+, or CD209.

As provided herein, the monocytes, HSCs, immature DCs, or mature DCscan be transduced with a viral vector, such as a lentivirus or an adenovirus or with mRNA encoding a desired protein. Any protocol for viral transduction can be used. For example, the cells can be cultured in a media cocktail. In some embodiments, the media comprises 100 ng/ml of mFlt3L/mTPO/mSCF, and 30 ng/ml of mIL-3. The cells can, for example, be cultured in this media incubate at 37° C. and 5% CO₂ for 24 hours or until activated. Following activation, the cells can be pre-treated with PGE2. The cells can then be transduced with the appropriate vector. In some embodiments, the virus is added at a MOI (multiplicity of infection) of 10, 100, or 100. After transductions, the cells can be isolated using, for example, beads or purification products that bind to one or more of the heterologous proteins encoded for by the vector. For example, cells can be collected after the transduction process using CD40L microbeads (Miltenyi) to purify the cells expressing the CD40L.

In some embodiments, to differentiate the transduced cells, such as C into CD14+CD16+ monocytes, the purified transduced cells can be expanded for 3-10 days by culturing 1×10⁵ CD34+ cells/ml in the expansion media in a G-Rex® 10M (X-VIVO™ 10 media; human AB serum 10%, rhSCF 50 ng/ml (R&D Systems), TPO 15 ng/ml (R&D Systems), IL-3 30 ng/ml (R&D Systems), Flt-3L 30 ng/ml (R&D Systems). After expansion of the cells (1-10 days), the cells can be moved into the differentiation medium in a G-Rex® 100M for 14 days. One non-limiting example of differentiation medium includes, but is not limited to, IMDM with 20% of human AB serum, SCF 25 ng/ml, M-CSF 30 ng/ml (R&D Systems), IL-3 30 ng/ml, and Flt-3L 30 ng/ml i. The cells can then be incubated in a differentiation cocktail for about, or at least 1, 2, 3, 4, 5, 6, 7, 8, 9, or 10 days. A non-limiting example of a differentiation cocktail is RPMI-1640; 3% human AB serum; GM-CSF 900 IU/ml; IL-4 1000 IU/ml; TNF-α 400 IU/ml; and TGF-β 0.2 ng/ml. The differentiation cocktail can enhance the differentiation of the monocytes into immature dendritic cells.

In some embodiments, to further enhance the maturation of the immature dendritic cells, the cells can be incubated in a maturation cocktail. In some embodiments, the maturation cocktail comprises an antigen, a tumor lysate or a live tumor cell. Examples of these are provided herein. The maturation cocktail can include, for example, GM-CSF 500 IU/ml; IL-15 400 ng/n1; IFN-γ 100 ng/ml; TNF-α 2 ng/ml; and PGE2 2 μg/ml. The amounts are exemplary only and are not limited to such amounts. Therefore, in some embodiments, the maturation composition (cocktail) comprises GM-CSF, IL-15, IFNγ−, TNF-α, and/or PGE2. The cells can incubated in this maturation compositions for about 12 to about 48 hours, about 20 to about 40 hours, about 30 to about 38 hours, about, or at least, 12, 16, 18, 20, 24, 28, 32, 36, 40, 44, or 48 hours. The maturation cocktail can also comprise TNF-α, IL-1β, IFN-α, IFN-γ, and/or polyinosine:polycytidylic acid (pIC).

Immature recombinant DCs were shown to exhibit phagocytotic ability The phagocytosis of fluorescently-labeled Escherichia coli particles by the immature DC product was observed by flow cytometry such that 86.13% of the product had phagocytosed the fluorescently labeled E. coli particles. The assays used can be any phagocytotic assay.

A useful aspect of the present compositions and methods includes the ability to bank the recombinant DCs to provide an off-the shelf vaccine for use at a local hospital, for ease of treating the patient. Such recombinant DCs, could then be handled in three different ways: 1) they could be loaded with autologous tumor lysate, or (2) in other instances they could be loaded with a tumor lysate; or (3) there could be no antigen loading in the case of non-resectable tumors.

In the next step of recombinant DC vaccine preparation, the recombinant alloDCs are matured with a cocktail of recombinant human cytokines such as those described above.

Then the cells are phenotyped and functionally evaluated, as well as to be evaluated for the expression of the relevant transgenes (e.g. CD40L, CXCL13, CD93, 4-1BBL, and IL-21). Finally, the recombinant DCs are frozen, cryopreserved, typically in a controlled rate cryopreservation and then utilized in a clinical setting after thawing. Such cells serve as the basis for the recombinant DC biological vaccine/immune adjuvant for treating cancers, tumors, and malignancies.

The cells can be thawed by thawing in a 37° C. water bath. In some embodiments, the cells are thawed without moving the cells in the water bath, that is motion or flicking. The cells can then be contacted with plasma and warmed thawing media. The cells can then be analyzed for viability before being administered to the subject. Examples of the markers are provided herein and above.

In some embodiments, the dendritic cells are matured with a cytokine cocktail and/or load the DC with tumor lysate. For example, the viable immature DCs can be cultured with tumor lysate. This can be done in the presence of a maturation cocktail, such as described above, or, for example, a composition that comprises TNF-α, IL-1β, IFN-α, IFN-γ, and pIC. The cells can be incubated with this composition for about 12 to about 36 hours, such as, or about, or at least, 20, 22, 24, 26, 28, or 30 hours. The mature cells can then be collected and analyzed. The mature cells can also be frozen using freezing media and stepwise freezing process, such as described herein.

As described herein, the cells can be incubated or loaded with a tumor lysate. The tumor lysate can be prepared in any manner. For example, in some embodiments, the tumor material is provided (isolated, obtained, resected, etc). The sample can be flash frozen in liquid nitrogen. In some embodiment, the sample is free of non-malignant tissue. This can be mean that the sample was removed without any non-malignant tissue or that the sample has been further processed to remove such non-malignant tissue. The sample can then be thawed and flash frozen 1-5 times to help lyse the cells. The lysate can be centrifuged and filtered to prepare the tumor lysate. The lysate can be stored frozen, for example, at −80° C. freezer.

The embodiments are now described with reference to the following examples. These examples are provided for the purpose of illustration only and the embodiments should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1

Production of Gene-Modified DCs from CD14+ Monocytes

Gene-modified DCs (five different gene modifications in Table 1) were produced. On Day 0, one (1) fresh full leukapheresis unit was processed on a CliniMACS Prodigy® device by running the LP14-MODC program with a TS510 tubing set. The cells were cultured in suspension culture at 37° C. in 5% CO₂. The program executed a CD14 labeling procedure followed by magnetic enrichment and positive fraction wash. The enriched target cells were sampled and analyzed for cell count, viability, and phenotype. A total of 1×10⁹ enriched target cells could be seeded into each Prodigy® chamber. On Day 2, a 50% media exchange occurred with Differentiation Medium (IL-4, GM-CSF). On Day 4, a 50% media exchange occurred with Differentiation Medium (IL-4, GM-CSF). On Day 6, a 50% media exchange occurred with Maturation Medium (IL-6, IL-1B, TNF-α, PGE2). On Day 7, cells were harvested out of the Prodigy® chamber. Cells were counted, analyzed, washed, and resuspended into electroporation buffer with appropriate combinations of respective mRNAs as depicted in Table 1. Electroporation occurred in cuvettes and were pooled within each respective condition. Electroporated cells were plated and incubated overnight. On Day 8, after overnight recovery, cells were harvested and formulated in Final Formulation Solution and CryoStor10, and cryogenically stored.

TABLE 1
Gene Modifications

| Group | Gene Modification |
|---|---|
| C1 | CD93/CXCL13/CD40L |
| C2 | CD93/CXCL13/4-1BBL |
| C3 | CD93/CXCL13/CD40L/IL-21 |
| C4 | CD93/CXCL13/4-1BBL/IL-21 |
| C5 | Unmodified DCs |

The distribution of cell types in the CD14 monocytes enriched from leukopaks on day 0 was evaluated by flow cytometry and the results are presented in Table 2. The mature MoDCs were also evaluated by flow cytometry on days 6 and 7 and the results presented in Table 3.

### Transfection Efficiency

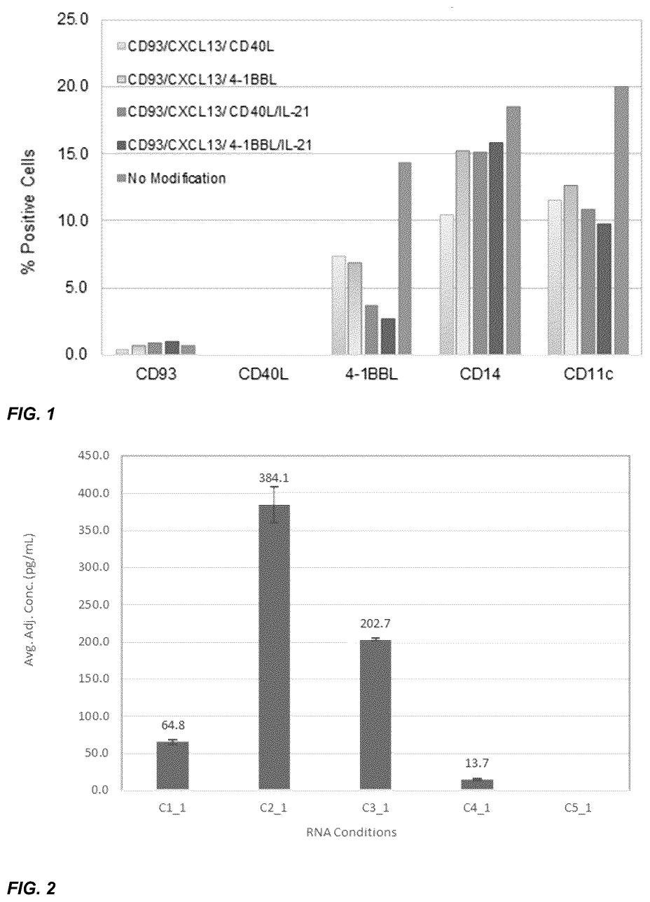

The transfection efficiency of the various gene modifications depicted in Table 1 are presented in FIG. 1. Transfection efficiency was low based on flow cytometry results.

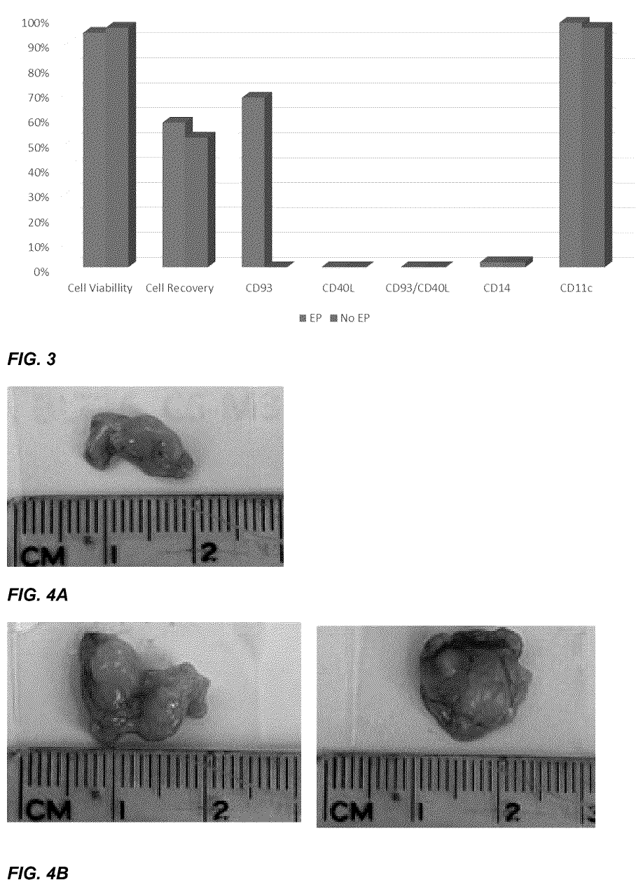

Transfection efficiency of the secreted protein CXCL13 was determined by measurement of secretion of CXCL13 from the gene-modified MoDCs by ELISA and the results are presented in FIG. 2.

These conclusions were further confirmed by the results of the functional assays.

TABLE 2
Distribution of cell phenotypes on Day 0.

| | | | | | | | | CD4+ | CD4− | | | | | CD56+ |
|---|---|---|---|---|---|---|---|---|---|---|---|---|---|---|
| Sample | Viable | CD3+ | CD3− | T | NKT | CD4+ | CD8+ | CD8+ | CD8− | | B | Neutro- | Eosino- | CD16+ |
| ID | cells | cells | cells | cells | cells | cells | cells | cells | cells | Monocytes | Cells | phils | phils | cells |
| Incoming | 97.84 | 45.18 | 54.82 | 40.78 | 4.4 | 24.19 | 14.22 | 0.71 | 1.65 | 28.13 | 15.37 | 0.96 | 0.07 | 7.57 |
| PE | 97.60 | 0.30 | 79.97 | 0.25 | 0.05 | 0.17 | 0.05 | 0.02 | 0.01 | 77.01 | 0.10 | 0.02 | 0.07 | 1.60 |
| Target | | | | | | | | | | | | | | |

Incoming refers to markers present on the source material cells; PE refers to cells post enrichment for CD14+ monocytes.

TABLE 3
Distribution of cell phenotypes at Day 6 and 7.

| Days | Sample ID | Viable | CD14 | CD209 | CD83 | CD40 | CD86 | HLA-ABC | HLA-DR |
|---|---|---|---|---|---|---|---|---|---|
| D 6 | D 6_Isotypes_Prodigy 1 | 94.52 | 0.55 | 0.70 | 0.51 | 0.69 | 0.60 | 0.73 | 0.75 |
| | D 6_MoDC Prodigy 1244 | 87.61 | 28.70 | 90.81 | 0.79 | 95.25 | 16.53 | 12.41 | 78.82 |
| | D 6_MoDC_Prodigy 1260 | 88.33 | 29.14 | 88.49 | 1.06 | 94.47 | 16.12 | 9.22 | 76.98 |
| | Mean | 87.97 | 28.92 | 89.65 | 0.93 | 94.86 | 16.33 | 10.82 | 77.90 |
| | Stdev | 0.51 | 0.31 | 1.64 | 0.19 | 0.55 | 0.29 | 2.26 | 1.30 |
| D 7 | D 7_Isotypies | 93.63 | 0.31 | 0.77 | 0.69 | 0.14 | 0.87 | 0.78 | 0.20 |
| | D 7_MoDC | 93.80 | 23.33 | 81.01 | 93.16 | 95.65 | 87.28 | 58.84 | 97.03 |

The Sample ID refers to different samples

### Functional Analysis of Gene-Modified MoDCs

A mixed lymphocyte reaction (MLR) assay was performed on the MoDCs. T cells from one donor proliferate in the presence of antigen presenting cells (APCs) from a different donor. The recognition of an HLA mismatch between two unrelated donors causes an immune response from the T cells. This assay determines if the transduced cells are functioning as expected.

T cells used in the assay were obtained from AllCells (1.56×10³ cells per well). The cells were co-cultured at MoDC:T cell ratios of 1:1 to 1:32 in 2-fold serial dilutions. The cells were co-cultured for seven days in MLR medium (RPMI1640 containing 5% human serum albumin, 2 mM L-glutamine, non-essential amino acids (1×) and 0.1 mM sodium pyruvate).

### Example 2

Expansion of DCs from CD34+ HSCs

The CD34+ cells were differentiated in mature DC cells by culturing them in appropriate culture flasks incubated at 37° C. with 5% CO₂. To differentiate from CD34+ cells to mature DC, different types of media with combinations of cytokines was used as described below. CD34+ cells, or HSCs (hematopoietic stem cells) were thawed from a frozen aliquot and were expanded in suspension culture for an optimized time-frame of 8 days in order to maximize cell yield while retaining at least 40% CD34+ cells by day 7. Culture was performed utilizing Cellgenix GMP SCGM (Stem Cell Growth Media) and a CD34+ expansion supplement from Stem Cells containing a proprietary composition of recombinant human cytokines, most notably: Flt3-L (fms-like tyrosine kinase 3 ligand), SCF (Stem Cell Factor), IL-3, (interleukin 3), IL-6 (interleukin 6), TPO (thrombopoietin). The media was refreshed every 2-3 days. CD34+ HSC cryopreserved at day 8 of expansion were thawed and cultured until day 23. The culture medium was changed to a monocyte differentiating IMDM medium containing 5% human AB serum, FLt3-L, IL-3, M-CSF, and SCF and the cells cultured for an additional 12 days to yield CD14+ monocytes. The CD14+ monocytes were then cultured for three days with AIM-V media containing 2% human AB serum, IL-4, and GM-CSF to yield immature DCs (imm-DCs). The imm-DCs were then cultured for 24 hr with 2% Human Serum, IL-4, GM-CSF, IL-6, IL-1b, IL-12, IL-15, PGE2, and TNF-α to produce mature DCs (MaDC). The cells were analyzed by flow cytometry at each step (Tables 4-8). For the purposes of this disclosure imm-DCs and MaDCs refer to DCs produced from HSCs.

The viability of the cells was above 90% and the cells were in good condition. The cells completely lost their CD34+ marker and were 96% positive for the DC marker, CD11c, confirming successful differentiation. The cells were 93% CD86 identifying successful definition of mature DC's from immature DC's. HLA-DR, HLA-ABC, CD40, and CD86 were all above 95% on average further confirming DC differentiation.

In conclusion, this procedure produced over 160 million DCs by the end of the cell culture experiment. HSC's (CD34+) successfully differentiated and were virtually absent from population starting Day 19 (at the end of monocyte differentiation).

At the end of HSC expansion (Day 8)/start of monocyte differentiation, CD14 was at 1.65% and by the end of monocyte differentiation phase was at 60% while 34% were CD11c+. The majority of the population successfully differentiated towards CD14 and superseded to pre-DC markers.

At the end of the MaDC production, CD14 was about 4% and CD11c averaged 95%, confirming MaDCs are dendritic cells and have successfully differentiated from monocytes.

The DCs generated in Example 1 were evaluated in vivo in the BLT26-MP2 murine tumor model of pancreatic cancer. Mice with established tumors were treated with DCs electroporated with mRNA or transduced with lentiviral vectors.

TABLE 4
The phenotypes of cells: Fresh CD34+
HSC thaw and expanded (Day 1-8)

| | Total | | | | | |
|---|---|---|---|---|---|---|
| | Viable | Total | | | | |
| | Cells | Cells | | | | |
| Day | (E6) | (E6) | Viability | CD34 | CD90 | CD45RA |
| 4 | 7.33 | 7.51 | 97.6% | 96.4% | 41.99% | 41.18% |
| 6 | 19.47 | 19.96 | 97.5% | 90% | 29.94% | 39.96% |
| 8 | 14.69 | 14.95 | 98.2% | 86.8% | 18.84% | 36.13% |

TABLE 5
The phenotypes of cells: From CD34+ to monocytes (Day 8 “rethaw” to Day 19)

| | Total Viable | Total Cells | | | | | | | | |
|---|---|---|---|---|---|---|---|---|---|---|
| Day | Cells (E6) | (E6) | Viability | CD14 | CD93 | CD56 | CD11c | CD19 | CD34 | CD40L |
| 8 | 7.47 | 7.90 | 94.5% | 1.65% | 1.37% | 0.24% | 1.77% | 0.03% | 88.28% | 0.46% |
| 10 | 11.58 | 12.66 | 91.5% | N/A | N/A | N/A | N/A | N/A | N/A | N/A |
| 12 | 30.3 | 31.63 | 95.8% | N/A | N/A | N/A | N/A | N/A | N/A | N/A |
| 15 | 82.16 | 85.26 | 96.4% | N/A | N/A | N/A | N/A | N/A | N/A | N/A |
| 17 | 119.33 | 123.66 | 96.5% | N/A | N/A | N/A | N/A | N/A | N/A | N/A |
| 19 | 118.4 | 126.4 | 93.7% | 59.65% | 43.16% | 0.42% | 33.7% | 0% | 3.92% | 1.17% |

TABLE 6
The phenotypes of cells: From monocytes (CD14) to immature DCs (Day 19-Day 22)

| | Total Viable | Total Cells | | | | | | | | |
|---|---|---|---|---|---|---|---|---|---|---|
| Day | Cells (E6) | (E6) | Viability | HLA-ABC | HLA-DR | CD209 | CD40 | CD86 | CD14*** | CD83 |
| 22 | 68.9 | 74.13 | 92.9% | 96.03% | 88.23% | 75.7% | 88.4% | 75.12% | 39.79% | 3.637% |

TABLE 7
The phenotypes of cells: From immature DCs to mature DCs frozen (Day 22-Day 23)

| | Total Viable | Total Cells | | | | | | | | |
|---|---|---|---|---|---|---|---|---|---|---|
| Day | Cells (E6) | (E6) | Viability | HLA-ABC | HLA-DR | CD209 | CD40 | CD86 | CD14 | CD83 |
| 23 | 149.16 | 163.08 | 91.5% | 95.7% | 96.3% | 63.2% | 98.71% | 97.61% | 38.35% | 93.1% |

TABLE 8
The phenotypes of cells: Mature DCs 48 hours after
thaw and electroporation (Day 23-Day 25)

| | Total | | | | | |
|---|---|---|---|---|---|---|
| | Viable | Total | | | | |
| | Cells | Cells | | Cells | | |
| Day | (E6) | (E6) | Viability | Recovered | CD14 | CD11c |
| 25 | 32E6 (EP) | 34E6 | 94% | 56% | 4% | 94% |
| 25 | 2.6E6 (No EP) | 2.7E6 | 96% | 52% | 4% | 96% |

### Example 3

Electroporation of HSC-Derived MaDCs with mRNA

MaDCs produced from CD34+ cells (HSC) in Example 2 were electroporated with three mRNAs encoding CD40L, CD93, and CXCL13. The surface marker expression of DC and monocyte markers was unchanged by electroporation. CD11c expression was 94% in electroporated cells vs. 96% in non-electroporated cells. CD14 expression was 4% in both electroporated and non-electroporated cells.

Results for expression various phenotype markers of electroporated cells are presented in FIG. 2. CD93 expression was measured by flow cytometry and expression of CD40L and CXCL13 were measured by ELISA.

CD40L was unable to be detected by flow cytometry and was determined via ELISA. CD93+/CD40L+ were absent and CD93 was found at 68%. CD14 was approximately 40% on Day 23 when the cells were harvested. After thawing and culturing for 24 hours, electroporating, then collecting after 24 hours, CD14 was about 4% with CD11c averaging at 95%. The final cells were confidently DC.

MaDCs electroporated with CD40L and CXCL13 were assayed for secretion of CD40L and CXCL13 as depicted in Tables 9 and 10.

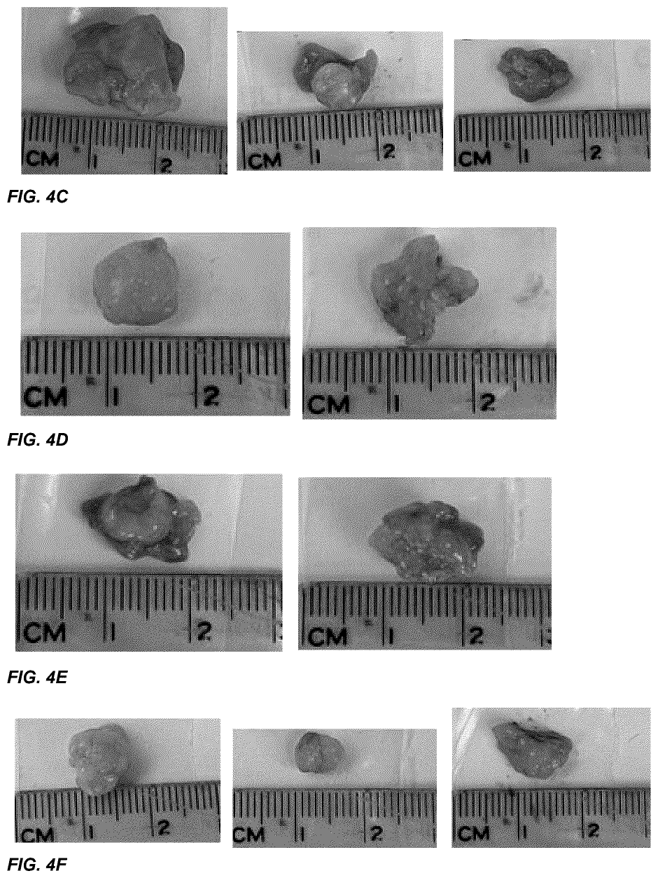

As depicted in Table 9, the control condition (no electroporation) tested negative for both CD40L and CXCL13. The electroporation (EP) condition showed CXCL13 and CD40L expression as determine by ELISA. The expression of CD93 was determined by flow cytometry. As depicted in FIG. 3, the expression of CD93 was negative in no electroporation condition and the expression of CD93 was over 65% in the electroporated condition. The results confirm the successful protein expression of CD93, CD40L and CXCL13 by electroporated mRNAs.

TABLE 9
ELISA

| | Vector | Cell | | | | | CD40L Conc | CXCL13 Conc |
|---|---|---|---|---|---|---|---|---|
| Sample | (μg) | Number | Living Cells | Total Cells | Viability | % Recovery | (pg/mL) | (pg/mL) |
| NEP | 0 | 5.50E+06 | 2.60E+06 | 2.70E+06 | 96% | 52% | 0 | 0 |
| EP | 1.5 | 5.50E+07 | 3.20E+07 | 3.40E+07 | 94% | 58% | 284.374 | 2845.154 |

NEP = no electroporation

TABLE 10
Phenotype of cells from thaw to Day 3. CD34+
cells expressing high levels of CD34, CD45, and
HLA-DR confirming optimal HSC characteristics.

| Surface | | | | | | | |
|---|---|---|---|---|---|---|---|
| Marker | CD34 | CD14 | CD209 | CD45 | HLA-DR | CCR7 | CD93 |
| Day 3 | 97.2% | 0% | 0% | 99.4% | 98.8% | 0% | 0% |

### Example 4

Expansion and LVV Transduction of CD34+ Cells and Differentiation into maDC

CD34+ cells, or HSC's, sourced from a frozen vial purchased or isolated onsite via Miltenyi CliniMACS from a G-CSF Mobilized Leukopak, were thawed and expanded in suspension culture for a total of 8 days while retaining at least 40% CD34+ by day 7. Culture was performed utilizing Cellgenix GMP SCGM™ (Stem Cell Growth Media) and a CD34+ expansion supplement from Stem Cells containing a proprietary composition of recombinant human cytokines most notably Flt3-L (fms-like tyrosine kinase 3 ligand), SCF (Stem Cell Factor), IL-3, (Interleukin 3), IL-6 (Interleukin 6), and TPO (Thrombopoietin). The media was refreshed every 2-3 days.

On Day 3, these cells were transduced with a construct with specific genes of interest, CD93, CD40L, and CXCL13, via a lentiviral vector (LV) generated by Vector Builder. The cells were plated on a retronectin dish and were plated with the same media for 2 hours with a PGE2 (prostaglandin 2) treatment and were then inoculated with a LentiBoost™ before introducing the LV on the retronectin dish. The transduction occurred for 24 hours followed by washing with medium before continuing to the 8 day cell expansion. Cells were analyzed via flow cytometry and were stained with monoclonal antibodies including anti-CD93 and anti-CD40L. The cells exhibit 30-40% of CD93 by day 8 and up to 15% CD40L. CD40L cleaves off the surface receptor CD40 and was found detectable via ELISA in the cell culture supernatant. CXCL13 was only detectable by ELISA and is found in the supernatant.

At the end of 8 day expansion, the culture medium was changed to a monocyte differentiating IMDM medium containing 5% human AB serum, FLt3-L, IL-3, M-CSF, and SCF and the cells cultured for an additional 12 days to yield CD14+ monocytes. The CD14+ monocytes were then cultured for three days with AIM-V media containing 2% human AB serum, IL-4, and GM-CSF to yield immature DCs (imm-DCs). The imm-DCs were then cultured for 24 hr with 2% Human Serum, IL-4, GM-CSF, IL-6, IL-1b, IL-12, IL-15, PGE2, and TNF-α to produce mature DCs (MaDC). The cells were analyzed by flow cytometry at each step (Tables 10-16). For the purposes of this disclosure, imm-DCs and MaDCs refer to DCs produced from HSCs.

As the cells differentiated towards monocytes, CD34 positivity was expected to decline significantly and CD14 positivity to increase. We observed nearly no CD34+ cells by Day 19 and moderate CD14+. As the monocytes differentiated towards immature DC's, the cells exhibited no CD34, moderate CD14 cells were observed (about 40-50%), high HLA-ABC, CD40, CD209, and CD86, and low CD83 and CCR7. After the cells were differentiated towards mature DC's, CD83, CD40, CD86, HLA-DR, and CCR7 should be significantly higher, and CD209 should be low between 20-50%. The morphology was also observed to ensure desired phenotype. Cells were round and had activated long tentacle-like phalanges. The cells expressed very little CD3, CD4, CD8, CD16, CD19, and CD56 suggesting little presence of other common cell sub-types which could be unintentionally differentiated during manufacturing process. The final cells retained High CD11c and at least 30% of CD93.

Cell expression was also analyzed via ELISA in order to detect excreted molecules in the supernatant. For this purpose, CD40L and CXCL13 were quantified on day 3, 8, and 23 in order to quantify gene expression before transduction, 72 hours after transduction, and at the end of the cell manufacturing process respectively. On day 3, the cells exhibited low CD40L at 0 pg/1×10⁶ cells and low CXCL13 expression at 0 pg/1×10⁶ cells. 72 hours later, after transduction of these genes, the cells expressed higher CD40L at 92.6 pg/1×10⁶ cells. By the end of the process, the CD40L population was partially retained on the cell surface and that secretion decreased appropriately back to 0 pg/1×10⁶ cells by end of process. The DS had also relatively high secretion 336.3 pg/1×10⁶ cells (Tables 17-18).

TABLE 11
Phenotype of cells from Day 3-8. CD34+ cells expressing
moderate levels of genes of interest while retaining
at least 40% CD34+ by end of expansion process

| Surface | | | | | | | |
|---|---|---|---|---|---|---|---|
| Marker | CD34 | CD14 | CD86 | CD45 | HLA-DR | CD93 | CD40L |
| Day 8 | 77.5% | 5% | 2.6% | 98.8% | 72.8% | 36% | 9% |

TABLE 12
Phenotype of cells from day 8-19 CD34+ cells undergo monocyte
differentiation and have lost their CD34+ stemness.

| Surface | | | | | | | |
|---|---|---|---|---|---|---|---|
| Marker | CD34 | CD14 | CD209 | CD40 | CD86 | CCR7 | HLA-DR |
| Day 19 | 1.053% | 23.25% | 1.52% | 10.81% | 9.18% | 7.32% | 42.95% |

TABLE 13
Phenotype of cells from day 19-22, monocytes
have differentiated to immature DC's

| Surface | | | | | | | |
|---|---|---|---|---|---|---|---|
| Marker | CD34 | CD14 | CD209 | CD40 | CD86 | CCR7 | HLA-DR |
| Day 22 | 0.07% | 26.25% | 24.68% | 36.94% | 18.26% | 10.56% | 55.74% |

TABLE 14
Day 23 Mature DC' cell pool exhibits few impurities

| Surface | | | | | | | |
|---|---|---|---|---|---|---|---|
| Marker | CD3 | CD45 | CD4 | CD16 | CD8 | CD56 | CD19 |
| Day 23 | 2.2% | 98.92% | 9.84% | 0.47% | 1.87% | 0.12% | 1.73% |

TABLE 15
Phenotype of cells on Day 23. 55%
mature DC pool in Drug substance

| Surface | | | | | | | HLA- |
|---|---|---|---|---|---|---|---|
| Marker | CD14 | CD34 | CD209 | CD40 | CD86 | CCR7 | DR |
| Day 23 | 50.25% | 0% | 18.9% | 58.47% | 47.81% | 55.11% | 50.98% |

TABLE 16
DC's have high CD11c by Day 23 and have retained
lenti-virally introduced genes of interest

| Surface Marker | CD11c | CD40L | CD93 |
|---|---|---|---|
| Day 23 | 91.995% | 7.865% | 31.07% |

TABLE 17
CD40L ELISA results on day 3, 8, and 23 of cell manufacturing process
ENOB DC-11 1.2 EXP1 D 3-D 23 CD40L
Results Normalized to Cell Number

| | Living | Cell Volume | CD40L | CD40L |
|---|---|---|---|---|
| Day | Cells | (mL) | (pg/mL) | (pg/1E6 cells) |
| D 3 | 7.48E+06 | 1.4 | 0 | 0 |
| D 8 | 2.82E+07 | 10.56 | 247.283 | 92.6 |
| D 23 | 2.10E+08 | 250 | 0 | 0 |

TABLE 18
CXCL13 ELISA results on day 3, 8, and 23 of cell manufacturing process
ENOB DC-11 1.2 EXP1 D 3-D 23 CXCL13
Results Normalized to Cell Number

| | Living | Cell Volume | CXCL13 | CXCL13 |
|---|---|---|---|---|
| Day | Cells | (mL) | (pg/mL) | (pg/1E6 cells) |
| D 3 | 7.48E+06 | 1.4 | 0 | 0 |
| D 8 | 2.82E+07 | 10.56 | N/A | N/A |
| D 23 | 2.10E+08 | 250 | 282.02 | 336.53 |

### Example 5

### Anti-Tumor Activity of Gene-Modified DCs

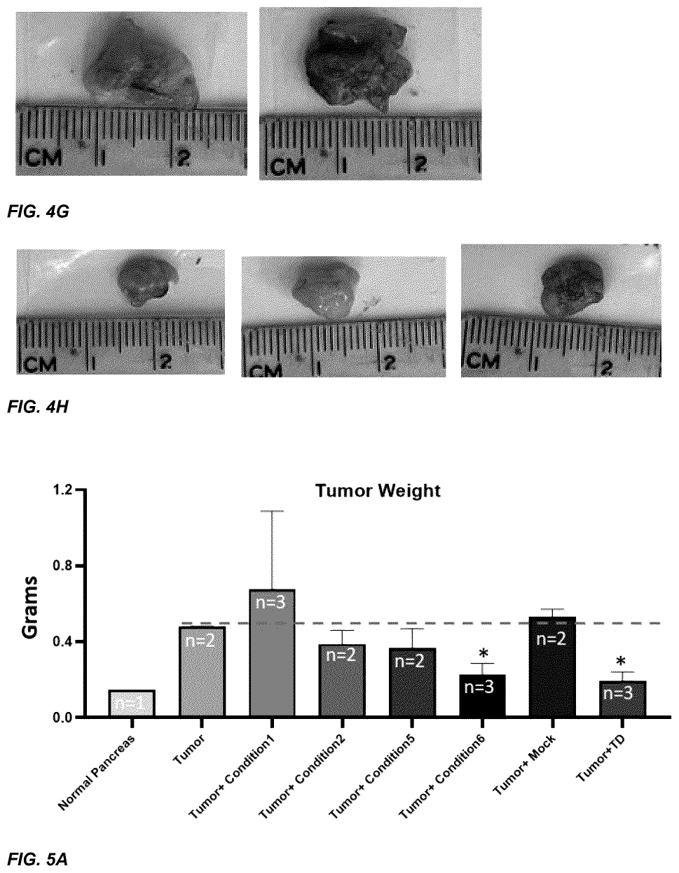

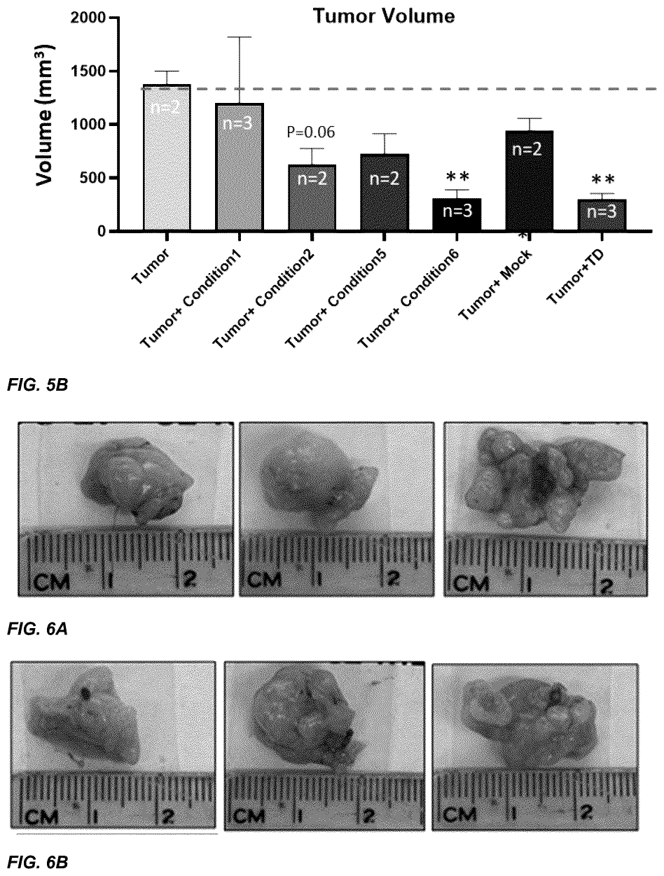

The DCs generated in Examples 1-4 were evaluated in vivo in the BLT26-MP2 murine tumor model of pancreatic cancer. Mice with established tumors were treated with DCs electroporated with mRNA or transduced with lentiviral vectors as described in FIGS. 4 and 5.

Gene-modified DCs (produced by lentiviral vector infection of HSC-derived DCs) were also pulsed with extracts from tumors and from cell lines and the activity of the antigen-pulsed cells evaluated in animals. Extracts from both monolayer tumor cells (pancreatic MP2 cells) and from samples of tumor tissue. The characteristics of the extracts are presented in Tables 19 and 20.

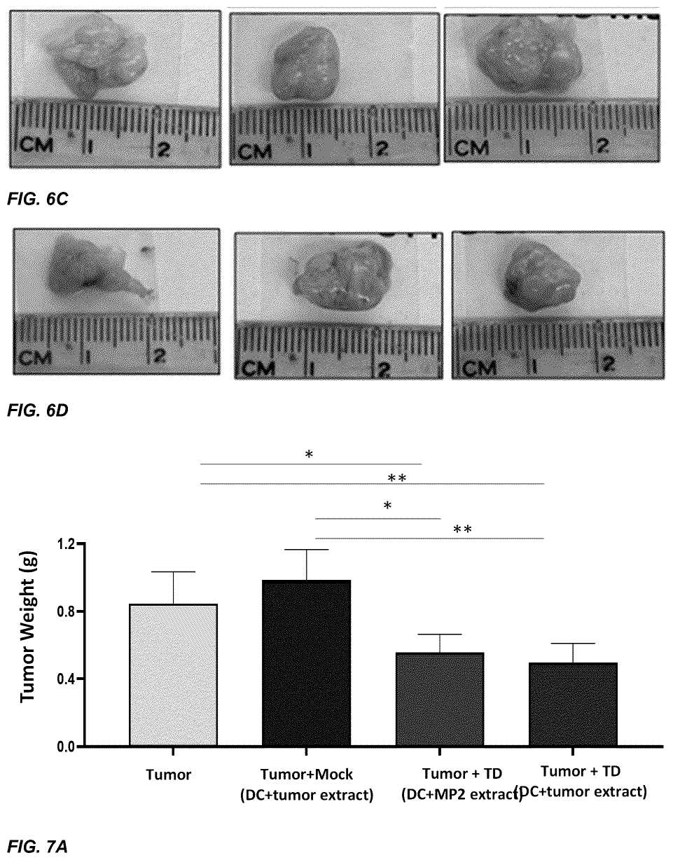

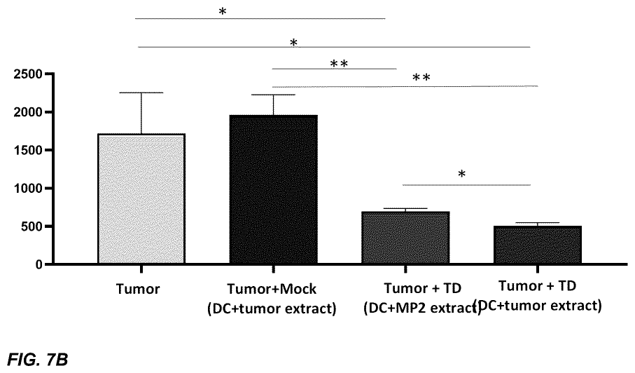

The recombinant DCs (produced by lentiviral vector infection of HSC-derived DCs) exposed to the tumor extract performed better than those exposed to cell extract as depicted in FIGS. 6 and 7 in NSG27 tumor-bearing mice. The mice were either untreated (tumor), MaDCs without gene-modification (Tumor+Mock), gene-modified MaDCs exposed to cell extract (DC+MP2 cell extract), or gene-modified MaDCs exposed to tumor extract (DC+tumor extract). The extract was prepared by rupturing the cells by sonication followed by multiple freeze thaw cycles. The protein was quantified and 18 μg of was added to each 1×10⁶ cells and cocultured for 15 to 18 hours. The protein quantification from each sample type is given in Tables 19 and 20.

TABLE 19
Tumor Lysate Total Protein Content Comparison

| | Tumor Weight | Protein | Volume | Protein |
|---|---|---|---|---|
| Sample | (g) | (mg/mL) | (mL) | (mg) |
| Sonicated | 0.18 | 6.2 | 0.89 | 5.52 |

TABLE 20
MP2 Lysate Total Protein content Comparison

| Sample | Cell Count | Protein (mg/mL) | Volume (μL) |
|---|---|---|---|
| MP2 Lysate Prep 2 | 2.5E+06 | 1.8 | 970 |

The mRNA-electroporated DCs (CD34+-derived) expressed the antigens and had anti-cancer activity similar to lentiviral transduced monocyte-derived DCs.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of producing gene-modified dendritic cells for cancer therapy comprising:
(a) obtaining CD34+ hematopoietic stem cells (HSCs);
(b) expanding the CD34+ HSCs;
(c) introducing one or more exogenous genes into the HSCs to produce gene-modified HSCs by infection with a viral vector comprising the genes;
(d) differentiating the gene-modified HSCs into monocytes;
(e) maturing the monocytes into immature dendritic cells (DCs);
(f) maturing the immature Dcs into mature dendritic cells (MaDCs) to produce gene-modified (recombinant) MaDCs; and
(g) harvesting the gene-modified MaDCs from the culture.

2. A method of producing gene-modified dendritic cells for cancer therapy comprising:
(a) obtaining CD34+ hematopoietic stem cells (HSCs);
(b) expanding the CD34+ HSCs;
(c) differentiating the gene-modified HSCs into monocytes;
(d) maturing the monocytes into immature dendritic cells (DCs);
(e) maturing the immature Dcs into mature dendritic cells (MaDCs) to produce gene-modified (recombinant) MaDCs;
(f) introducing one or more exogenous genes into the HSCs to produce gene-modified HSCs by electroporation of mRNA encoding the genes; and
(g) harvesting the gene-modified MaDCs from the culture.

3. The method of either of claim 1 or 2, further comprising pulsing the gene-modified MaDCs with tumor antigens.

4. The method of claim 3, wherein the tumor antigens are present in at least one tumor cell lysate.

5. The method of claim 4, wherein the tumor cell lysate is prepared from a tumor or a tumor cell line.

6. The method of claim 4, wherein the tumor cell lysate is allogeneic or autologous to the antigen activated dendritic cell.

7. The method of claim 3, wherein the tumor antigens comprise a tumor tissue sample, a tumor cell line, a purified tumor antigen, or a synthetic tumor antigen.

8. The method of either of claim 1 or 2, wherein the immature dendritic cells are characterized by expression of CD209, HLA-DR, CD40, CD86 and CD14 and low expression of CCR7.

9. The method of either of claim 1 or 2, wherein the mature dendritic cells are characterized by expression of CD1a, CCR7, HLA-DR and CD83 and low expression of CD14

10. The method of any one of claims 1-9, wherein the one or more exogenous genes are selected from CD93, CXCL13, CD40L, IL-21, and 4-1BBL.

11. The method of claim 10, wherein the one or more exogenous genes comprise two genes, three genes, four genes, or five genes.

12. The method of any one of claims 1-11, further comprising cryopreserving the cells after one or more of steps (a)-(f).

13. A method of treating cancer, comprising administering to a subject in need thereof the gene-modified MaDCs prepared according to the methods of any one of claims 1-12.

14. The method of claim 13, wherein the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma/colorectal cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

15. The method of claim 13 or 14, wherein the route of administration is via intratumoral, peritumoral, intradermal, subcutaneous, intramuscular, or intraperitoneal, injection.

16. The method of any one of claims 13-15, wherein the administering is via subcutaneous, intratumoral or intradermal injection.

17. The method of any of claims 13-16, wherein the gene-modified MaDCs are cryopreserved and thawed prior to administration.

18. The method of claim 13, wherein the MaDCs are not pulsed with an antigen prior to administration.

19. A kit comprising gene-modified MaDCs prepared by the methods of any one of claims 1-12, wherein the gene-modified MaDC are frozen or cryopreserved in a container, and optionally comprising a cryopreserved allogeneic tumor lysate in a separate container.

20. The kit of claim 19 wherein the gene-modified MaDC or composition is frozen in a container.

21. The kit of claim 19 or 20, further comprising separate containers comprising one or more buffers, and optionally comprising one or more activation agents.

22. The kit of any one of claims 19-21, further comprising instructions for incubating and/or processing the dendritic cells or compositions.

23. A method of treating cancer in a subject comprising administering to the subject gene-modified MaDCs prepared by the methods of any one of claims 1-12, wherein the gene-modified MaDCs or composition has not been pulsed with a tumor antigen.

24. A method of treating cancer in a subject comprising administering to the subject gene-modified MaDCs prepared by the methods of any one of claims 1-12, wherein the gene-modified MaDCs has been pulsed with a tumor antigen or lysate.

25. A method of activating the immune system, comprising administering to a subject in need thereof an effective amount of gene-modified MaDCs prepared by the methods of any one of claims 1-12.


SEQUENCE LISTING (10 sequences)

SEQ ID NO: 1 - DNA, 786 bp, Homo sapiens
  source 1..786: mol_type = unassigned DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 2 - DNA, 327 bp, Homo sapiens
  source 1..327: mol_type = unassigned DNA
atgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccc

SEQ ID NO: 3 - DNA, 1956 bp, Homo sapiens
  source 1..1956: mol_type = unassigned DNA
atggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgtcctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgtgctgcaacagccagtcccacaaggggccccgagggcacccccaaggctacacccaccacaagtagaccttcgctgtcatctgacgcccccatcacatctgccccactcaagatgctggcccccagtgggtccccaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgc

SEQ ID NO: 4 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 6 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 7 - DNA, 762 bp, Homo sapiens
  source 1..762: mol_type = unassigned DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 8 - DNA, 489 bp, Homo sapiens
  source 1..489: mol_type = unassigned DNA
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 9 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS